(19) United States
(12) Patent Application Publication
(10) Pub. No.: US 2016/0319035 A1
(43) Pub. Date: Nov. 3, 2016

(54) PDK1 BINDING MOLECULES AND USES THEREOF

(71) Applicants: Scott Kendall Dessain, Wynnewood, PA (US); Baron Christopher Heimbach, Havertown, PA (US)

(72) Inventors: Scott Kendall Dessain, Wynnewood, PA (US); Baron Christopher Heimbach, Havertown, PA (US)

(21) Appl. No.: 14/961,492

(22) Filed: Dec. 7, 2015

Related U.S. Application Data
(60) Provisional application No. 62/088,530, filed on Dec. 5, 2014.

(51) Int. Cl. C07K 16/40; G01N 33/574; C07K 14/705; C07K 16/30; A61K 39/395; C07K 14/725

(52) U.S. Cl. CPC C07K 16/40; A61K 39/39558; C07K 14/7051; C07K 14/70517; C07K 14/70521; C07K 16/3015; G01N 33/57488; C12Y 207/11002; C07K 2317/565; C07K 2317/56; C07K 2317/73; C07K 2319/03; C07K 2319/02; C07K 2317/622; C07K 2317/53; C07K 2317/732; C07K 2317/734; A61K 2039/505

(57) ABSTRACT

The present invention is based, at least in part, on the discovery that PDK1 is associated with the outer plasma membrane of the cell. Accordingly, the present invention encompasses 3-phosphoinositide-dependent protein kinase-1 (PDK1) binding molecules, such as an antibody. The binding molecules of the invention are useful for detecting PDK1 and for inhibiting PDK1 activity, e.g., in a human subject having a disorder in which PDK1 activity is detrimental, such as a cancer.

### RELATED APPLICATIONS

This application claims priority to U.S. Provisional Patent Application No. 62/088,530 filed on Dec. 5, 2014, the contents of which are incorporated by reference herein.

### SEQUENCE LISTING

The instant application contains a Sequence Listing which has been submitted electronically in ASCII format and is hereby incorporated by reference in its entirety. Said ASCII copy, created on Dec. 7, 2015, is named 125201-00320_SL.txt and is 36,072 bytes in size.

### FIELD OF THE INVENTION

The present invention relates to 3-phosphoinositide-dependent protein kinase-1 (PDPK1 which is also referred to as PDK1) binding molecules (e.g. antibodies, antigen binding fragments thereof, and polypeptides) and their use in the diagnosis and/or treatment of cancer.

### BACKGROUND

Cancer is presently one of the leading causes of death in developed nations and is a major health problem of modern societies. For example, 1 in 8 U.S. women (12%) will develop breast cancer during their lifetime and in 2014 over 232,000 U.S. women will be newly diagnosed with breast cancer. Metastatic breast cancer is diagnosed in 6-10% of new breast cancer cases, however, 20-30% of all breast cancer cases will become metastatic (O'Shaughnessy, 2005, Oncologist 10 Suppl 3: p. 20-9; ACS, Breast Cancer Facts & Figures 2013-2014, 2013, American Cancer Society, Inc.: Atlanta). Metastatic breast cancer survival is estimated at 18 to 24 months with 5-year and 10-year survival rates of 24% and 5%, respectively (Rahman, et al., 1999, Cancer 85(1): p. 104-11; Greenberg, 1996, J Clin Oncol, 1996. 14(8): 2197-205). Metastatic breast cancer treatment frequently includes hormone therapy and/or chemotherapy with or without trastuzumab. Patients with limited symptomatic metastases may be treated with radiation and/or surgery (NCI. PDQ Breast Cancer Treatment. National Cancer Institute 2014 Nov. 14, 2014; available from cancer.gov/cancertopics/pdq/treatment/breast/healthprofessional/page6). Despite advances in survival rates for women with metastatic breast cancer, cures are rare and palliative therapy is the treatment aim (NCI. PDQ Breast Cancer Treatment, cited above).

A critical obstacle to the implementation of new monoclonal antibody (mAb) immunotherapies is the limited number of available cancer-specific antigen targets. New, cancer-specific antigen targets are needed to improve metastatic breast cancer treatment. Thousands of potentially antigenic changes occur during tumorigenesis: mutated proteins, over-expressed or mis-localized proteins, altered glycosylation, and splice isoform changes (Eifert et al., 2012, Nat Rev Cancer 12(8): p. 572-8). Many of these antigenic changes are the result of the Epithelial Mesenchymal Transition (EMT) a phenotypic shift associated with metastasis and the cancer stem cell phenotype (Kalluri, et al., 2009, J Clin Invest 119(6): p. 1420-8; Shapiro, et al., 2011, PLoS Genet 7(8): p. e1002218; Dittmar et al., 2012, Mol Cell Biol 32(8): p. 1468-1482; Maupin, 2010, PLoS ONE 5(9): p. e13002). In the EMT, epithelial cancer cells acquire the mesenchymal qualities of motility, invasiveness, and metastasis. Cells that acquire EMT characteristics express a vast diversity of antigens that differ from those expressed by their normal progenitors. These antigenic changes may be interpreted as non-self by the cancer patient immune system, however clinical medicine has not yet capitalized on these discoveries (Eifert et al., 2012, Nat Rev Cancer 12(8): p. 572-8).

Often immunotherapy target discovery methods rely on the murine immune response to detect new antigens on injected human cancer cells (Rust, et al., 2013, Mol Cancer 12: 11.). Yet, the key challenge is not just to identify the antigen, but to find the “target within the target,” a protein epitope that is antigenically different from all other normal epitopes in the body. This exclusivity cannot be predicted empirically from structural analyses.

Thus, new therapies directed at cancer-specific antigen targets are needed for treatment of metastatic cancers and to improve survival outcomes.

### SUMMARY OF THE INVENTION

The present invention is based, at least in part, on the discovery that PDK1 is associated with the outer plasma membrane of the cell.

The present invention relates, at least in part, to 3-phosphoinositide-dependent protein kinase-1 (PDK1) binding molecules, including anti-PDK1 antibodies, or antigen-binding fragments thereof, that bind PDK1, e.g., the extracellular portion of PDK1 or to a cell comprising extracellular PDK1, and uses thereof. Various aspects of the invention relate to anti-PDK1 antibodies and antibody fragments, and pharmaceutical compositions thereof. In one embodiment, the anti-PDK1 antibody, or antigen-binding fragment thereof, is a 2B9 antibody, as further described herein.

Accordingly, in a first aspect, the present invention features a binding molecule which binds to extracellular 3-phosphoinositide-dependent protein kinase-1 (PDK1).

In another aspect, the present invention features a binding molecule which binds to a cell expressing extracellular 3-phosphoinositide-dependent protein kinase-1 (PDK1) and does not bind to a cell which does not express extracellular PDK1.

In one embodiment, the binding molecule inhibits migration of a cancer cell expressing extracellular PDK1. In another embodiment, the binding molecule is capable of modulating a biological activity of PDK1.

In one embodiment, the binding molecule is an antigen binding protein or polypeptide. In another embodiment the binding molecule is an antibody, or antigen binding fragment thereof. In a further embodiment, the antibody, or antigen binding fragment thereof, is a recombinant human antibody, or antigen binding fragment thereof.

In a related embodiment, the antibody, antigen binding fragment thereof, or polypeptide is expressed in a mammalian cell. In another related embodiment, the antibody, antigen binding fragment thereof, or polypeptide is expressed in a eukaryotic or prokaryotic cell. In a further embodiment, the antibody, antigen binding fragment thereof, or polypeptide is synthesized in a cell free system.

In one embodiment, the antibody, or antigen binding fragment thereof, comprises a heavy chain variable region comprising a CDR3 having the amino acid sequence of SEQ ID NO: 7 and a light chain variable region comprising a CDR3 having the amino acid sequence of SEQ ID NO: 14. In another embodiment, the antibody, or antigen binding fragment thereof, comprises a heavy chain variable region comprising a CDR2 having the amino acid sequence of SEQ ID NO: 6 and a light chain variable region comprising a CDR2 having the amino acid sequence of SEQ ID NO: 13. In another embodiment, the antibody, or antigen binding fragment thereof, comprises a heavy chain variable region comprising a CDR1 having the amino acid sequence of SEQ ID NO: 5 and a light chain variable region comprising a CDR1 having the amino acid sequence of SEQ ID NO: 12. In a further embodiment, the antibody, or antigen binding fragment thereof, is an IgG isotype.

In one embodiment, the antibody, or antigen binding fragment thereof, comprises a heavy chain variable region comprising the amino acid sequence of SEQ ID NO: 4. In one embodiment, the antibody, or antigen binding fragment thereof, comprises a light chain variable region comprising the amino acid sequence of SEQ ID NO: 11. In one embodiment, the antibody, or antigen binding fragment thereof, comprises a heavy chain variable region comprising the amino acid sequence set forth in SEQ ID NO: 4.

In one embodiment, the binding molecule is the antibody 2B9.

In another embodiment, the binding molecule is a small molecule.

In another embodiment, the binding molecule is conjugated to an imaging agent.

In one embodiment, the present invention features a pharmaceutical composition comprising the binding molecule of any one of the aspects described herein, and a pharmaceutically acceptable carrier.

In another embodiment, the present invention features a method of treating a cancer in a subject in need thereof comprising administering to the subject the binding molecule of any of the aspects or embodiments described herein, thereby treating the cancer in the subject.

In another embodiment, the present invention features a method of treating a cancer in a subject comprising (a) selecting a subject having cancer, wherein the cancer is characterized by expression of extracellular 3-phosphoinositide-dependent protein kinase-1 (PDK1); and (b) administering to the subject a therapeutically effective amount of the binding molecule of any of the aspects or embodiments described herein.

In one embodiment, the invention features a method of treating a cancer in a subject in need thereof comprising (a) obtaining a sample from a subject having cancer; (b) testing the sample for expression of extracellular 3-phosphoinositide-dependent protein kinase-1 (PDK1); and (c) administering to the subject a therapeutically effective amount of the binding molecule of any of the aspects or embodiments described herein, when extracellular PDK1 is present in the sample.

In one embodiment, the invention features a method of treating a cancer in a subject in need thereof comprising (a) obtaining a sample from a subject having cancer; (b) testing the sample for extracellular 3-phosphoinositide-dependent protein kinase-1 (PDK1); and (c) administering to the subject a therapeutically effective amount of the binding molecule of any of the aspects or embodiments described herein, when extracellular PDK1 is present in the sample.

In one embodiment, the binding molecule is an antigen binding protein and does not cross the cell membrane.

In one aspect, the present invention features a method of treating a cancer in a subject in need thereof comprising (a) obtaining a sample from a subject having cancer; (b) testing the sample for expression of extracellular 3-phosphoinositide-dependent protein kinase-1(PDK1); and (c) administering to the subject a therapeutically effective amount of a PDK1 inhibitor.

In one aspect, the present invention features a method of treating a cancer in a subject in need thereof comprising (a) obtaining a sample from a subject having cancer; (b) testing the sample for extracellular 3-phosphoinositide-dependent protein kinase-1(PDK1); and (c) administering to the subject a therapeutically effective amount of a PDK1 inhibitor.

In one embodiment, the cancer is metastatic or non-resectable. In a further embodiment, the cancer is selected from breast cancer, lung cancer, prostate cancer, acute myeloid leukemia (AML), cervical cancer and squamous cell carcinoma.

In another embodiment, the method further comprises administering to the subject at least one additional therapeutic agent. In a further embodiment, the additional therapeutic agent is an agent selected from the group consisting of an anti-angiogenic agent, an anti-neoplastic composition, a chemotherapeutic agent, and a cytotoxic agent.

In one embodiment, the present invention features a method for determining malignancy of a tumor from a subject comprising determining the presence of extracellular 3-phosphoinositide-dependent protein kinase-1 (PDK1) in a sample from a tumor from the subject by contacting the sample with the binding molecule of any of the aspects or embodiments described herein, wherein the presence of extracellular PDK1 as detected by the binding molecule in the sample indicates that the tumor is malignant.

In another embodiment, the present invention features a method for determining whether a cancer in a subject is metastatic, said method comprising determining the presence of extracellular 3-phosphoinositide-dependent protein kinase-1 (PDK1) in a sample from the subject by contacting the sample with the binding molecule of any of the aspects or embodiments herein, wherein the presence of extracellular PDK1 as detected by the binding molecule in the sample indicates that the cancer is metastatic.

In another embodiment, the present invention features a method of identifying a subject having cancer which is responsive to anti-PDK1 treatment comprising determining the presence of extracellular 3-phosphoinositide-dependent protein kinase-1 (PDK1) in a sample from the subject by contacting the sample with the binding molecule of any of the embodiments or aspects herein, wherein the presence of extracellular PDK1 as detected by the binding molecule in the sample indicates that cancer will be responsive to an anti-PDK1 treatment.

In another embodiment, the invention features a method of identifying a subject having cancer which is responsive to the binding molecule of any of the aspects or embodiments herein, said method comprising determining the presence of extracellular 3-phosphoinositide-dependent protein kinase-1 (PDK1) in a sample from the subject, wherein the presence of extracellular PDK1 in the sample indicates that cancer will be responsive to treatment with the antigen binding protein.

In one embodiment, the cancer is metastatic or non-resectable. In another embodiment, the cancer is selected from the group consisting of breast cancer, lung cancer, prostate cancer, acute myeloid leukemia (AML), cervical cancer and squamous cell carcinoma.

In one embodiment, the present invention features a method of detecting extracellular 3-phosphoinositide-dependent protein kinase-1 (PDK1) in a sample, comprising contacting the sample with the binding molecule of any of the aspects or embodiments herein.

In another aspect, the present invention features an isolated anti-PDK1 antibody, or antigen binding fragment thereof, comprising a heavy chain variable region comprising a CDR1 having an amino acid sequence of SEQ ID NO: 5, a CDR2 having an amino acid sequence of SEQ ID NO: 6, and CDR3 having an amino acid sequences of SEQ ID NO: 7, and a light chain variable region comprising a CDR1 having an amino acid sequence of SEQ ID NO: 12, a CDR2 having an amino acid sequence of SEQ ID NO: 13, and CDR3 having an amino acid sequences of SEQ ID NO: 14.

In one embodiment, the antibody, or antigen binding fragment is a recombinant human antibody. In another embodiment, the antibody, or antigen binding fragment thereof, is an IgG isotype.

In another aspect, the present invention features an isolated anti-PDK1 antibody, or antigen binding fragment thereof, comprising a heavy chain variable region comprising an amino acid sequence of SEQ ID NO: 4, and a light chain variable region comprising an amino acid sequence of SEQ ID NO: 11.

In one embodiment, the antibody, or antigen binding fragment is a recombinant human antibody. In another embodiment, the antibody, or antigen binding fragment thereof, is an IgG isotype.

In one embodiment, the anti-PDK1 antibody, or antigen binding fragment thereof, of any of the above aspects or embodiments binds to extracellular membrane bound 3-phosphoinositide-dependent protein kinase-1 (PDK1).

In one embodiment, the anti-PDK1 antibody, or antigen binding fragment thereof, of any of the above aspects or embodiments binds to a cell expressing extracellular PDK1.

In one embodiment, the anti-PDK1 antibody, or antigen binding fragment thereof, of any of the above aspects or embodiments inhibits migration of a cell expressing extracellular PDK1.

In one embodiment, the anti-PDK1 antibody, or antigen binding fragment thereof, of any of the above aspects or embodiments is capable of modulating a biological activity of PDK1.

In one embodiment, the anti-PDK1 antibody, or antigen binding fragment thereof, of any of the above aspects or embodiments is conjugated to an imaging agent.

In another embodiment, the invention present features a pharmaceutical composition comprising the anti-PDK1 antibody, or antigen binding fragment thereof, of any one of the above aspects or embodiments, and a pharmaceutically acceptable carrier.

In another embodiment, the present invention features a method for reducing cell migration and invasion, the method comprising the step of contacting a cell expressing extracellular PDK1 with the antibody, or antigen-binding portion thereof, of any of the above aspects or embodiments, such that cell migration and invasion is reduced.

In another embodiment, the invention features a method for reducing PDK1 activity, the method comprising the step of contacting a cell expressing extracellular PDK1 with the antibody, or antigen-binding portion thereof, of any one of the above aspects or embodiments, such that PDK activity is reduced.

In one embodiment, the method is in vivo.

In another embodiment, the invention features a method for treating a human subject having a disorder in which extracellular expression of PDK1 is detrimental comprising administering an effective amount of the anti-PDK1 antibody, or antigen binding portion thereof, of any one of the above aspects or embodiments to the subject.

In one embodiment, the disorder is cancer. In a further embodiment, the cancer is metastatic or non-resectable cancer. In another further embodiment, the cancer is selected from the group consisting of breast cancer, lung cancer, prostate cancer, acute myeloid leukemia (AML), cervical cancer, and squamous cell carcinoma. In another embodiment, the disorder is a solid tumor or a hematopoietic cancer.

In another embodiment, the method of any of the above embodiments further comprises administering an additional therapeutic agent to the subject. In a related embodiment, the additional therapeutic agent is an agent selected from the group consisting of an anti-angiogenic agent, an anti-neoplastic composition, a chemotherapeutic agent, and a cytotoxic agent.

In another embodiment, the method of any of the above embodiments further comprises administering radiation to the subject.

In another embodiment, the present invention features a method of treating a cancer in a subject comprising (a) selecting a subject having cancer, wherein the cancer is characterized by expression of extracellular PDK1; and (b) administering to the subject a therapeutically effective amount of the anti-PDK1 antibody, or antigen binding portion thereof, of any of the above aspects or embodiments.

In another aspect, the present invention features a nucleic acid encoding a chimeric antigen receptor (CAR), wherein the CAR comprises an extracellular binding domain capable of binding to extracellular PDK1, a transmembrane domain, and an intracellular signaling domain.

In one embodiment, the extracellular binding domain is an anti-PDK1 antibody, or an antigen binding fragment thereof. In another embodiment, the antibody, or antigen binding fragment thereof, comprises a heavy chain variable region comprising a CDR1 having an amino acid sequence of SEQ ID NO: 5, a CDR2 having an amino acid sequence of SEQ ID NO: 6, and CDR3 having an amino acid sequence of SEQ ID NO: 7, and comprises a light chain variable region comprising a CDR1 having an amino acid sequence of SEQ ID NO: 12, a CDR2 having an amino acid sequences of SEQ ID NO: 13, and CDR3 having an amino acid sequence of SEQ ID NO: 14. In another further embodiment, the antibody, or antigen binding fragment thereof, comprises a heavy chain variable region comprising an amino acid sequence of SEQ ID NO: 4, and a light chain variable region comprising an amino acid sequence of SEQ ID NO: 11.

In one embodiment, the antigen binding fragment is an scFv.

In another embodiment, the intracellular signaling domain of the nucleic acid comprises a CD3 zeta signaling domain. In another further embodiment, the nucleic acid further comprises a co-stimulatory signaling domain.

In one embodiment, the present invention features a vector comprising the nucleic acid sequence of any of the aspects or embodiments described herein. In a further embodiment, the vector is a viral vector. In a related embodiment, the viral vector is a lentiviral vector or a retroviral vector.

In one embodiment, the present invention features an immune effector cell comprising the vector of any of the embodiments described herein. In a further embodiment, the immune effector cell is a T lymphocyte. In a related embodiment, the immune effector cell is obtained from a subject having cancer.

In another embodiment, the present invention features a method of treating cancer in a subject in need thereof, comprising administering to the subject the immune effector cell of any of the aspects or embodiments described herein. In a further embodiment, the cancer is breast cancer, lung cancer, prostate cancer, acute myeloid leukemia (AML), cervical cancer and squamous cell carcinoma. In another further embodiment, the cancer is a solid tumor or a hematopoietic cancer.

In one aspect, the present invention features an isolated anti-PDK1 human antibody, or antigen binding portion thereof, produced by hybridoma cell line 2B9 (ATCC Accession No. ______).

In one aspect, the present invention features the hybridoma cell line 2B9 deposited under ATCC Accession No. ______.

### BRIEF DESCRIPTION OF THE DRAWINGS

FIG. 1A shows 2B9 binding to three breast cancer cell lines (MCF7, MDA-MB-231, T47D) and one cervix squamous cell carcinoma cell line (A431).

FIG. 1B shows a Retrogenix 293T cell gene expression array demonstrating that monoclonal antibody (mAb) 2B9 binds to PDK1. The left panel shows the positive control GFP expression and the right panel shows duplicate signals of 2B9 binding PDK1 (PDPK1) transfected domains. FCGR1A is CD64, the high-affinity IgG Fc receptor 1.

FIG. 2A provides the results of cells that were transfected with myc-tagged PDK1 and subsequently assessed by flow cytometry to determine whether PDK1 is detectable on the outer plasma membrane of live stained cells.

FIG. 2B provides the results of cells that were transfected with myc-tagged PDK1 and subsequently assessed by flow cytometry to determine whether PDK1 is detectable on the outer plasma membrane of fixed, permabilized cells. As described in FIG. 2A and FIG. 2B, antibody 2B9 and anti-myc antibodies were able to bind PSK1 on the outer plasma membrane.

FIG. 3 shows assessment of 2B9 by sandwich ELISA to confirm the identity of PDK1. FIG. 4 shows binding of the 2B9 mAb to recombinant PDK1 (baculovirus sf9 source) as determined by ELISA assay. The 2B9 antibody was expressed by the 2B9 hybridoma cell line and purified from the hybridomas cell culture supernatant using Protein G affinity chromatography.

FIG. 5 shows cell surface expression of PDK1 on transfected 293T cells. DNAs containing different PDPK1 cDNAs were transfected into 293T cells and surface expression of 2B9 antigen (PDPK1) was analyzed by flow cytometry on live (unpermeabilized) cells. The constructs also encoded an N-terminal myc tag, enabling detection with the 9E10 mAb (aMyc). Transfected genes encoded wild-type, kinase dead (K111A), autophosphorylation site dead (S241A), phosphatidyl serine pocket mutated (K465A, R466A, K467A) and pleckstrin-homology (PH) domain mutated (R472A, R473A, R474A) forms.

FIG. 6 provides Western blot data showing that mutant myc labeled PDK1 proteins are expressed by transfected cells.

FIG. 7 shows a summary of binding experiments identifying which regions of the PDK1 protein are bound by anti-PDK1 antibodies known in the art.

FIG. 8 shows the results of experiments to determine outer plasma membrane PDK1 expression on K562 cells using anti-PDK1 antibodies known in the art (referenced in FIG. 7).

FIG. 9 provides results of experiments showing expression of PDK1 on K562 cells.

FIG. 10 shows reduced cell migration of MDA-MB-231 cells (a human breast epithelial adenocarcinoma cell line that is metastatic) treated with mAb 2B9 in an in vitro cell migration assay. The cells were grown to 80% confluence then were serum starved in the presence or absence of antibodies 6A or 2B9 at 10 ug/ml. 24 hours later, MDA-MB-231 cells from each of the three treatment groups were removed from the culture vessels and counted. 50,000 MDA-MB-231 cells from each group were re-suspended in serum free L-15 medium+/−10 ug/ml 6A or 2B9 then seeded onto 24-well Matrigel Invasion chambers (Corning) (n=3). Images of invasion chambers for the negative control (A), Antibody 6A (B), and Antibody 2B9 (C) were captured at 100× magnification.

### DETAILED DESCRIPTION

The present invention relates, at least in part, to 3-phosphoinositide-dependent protein kinase-1 (PDK1) binding molecules, including anti-PDK1 antibodies, or antigen-binding fragments thereof, that bind PDK1, e.g., the extracellular portion of PDK1 or to a cell comprising extracellular PDK1, and uses thereof. Various aspects of the invention relate to anti-PDK1 antibodies and antibody fragments, and pharmaceutical compositions thereof. In one embodiment, the anti-PDK1 antibody, or antigen-binding fragment thereof, is a 2B9 antibody, as further described herein.

In another aspect, methods of using the PDK1 binding molecules (e.g. antibodies) of the invention to prevent or treat cancer, including, but not limited to breast cancer, cervical cancer and squamous cell carcinoma, in a subject, are also encompassed by the invention. The invention also encompasses methods for diagnosing or prognosing cancer in a subject, as well as methods for determining malignancy of a tumor and methods for determining whether a cancer is metastatic, using the PDK1 binding molecules of the invention (e.g. anti-PDK1 antibodies, or antigen-binding fragments thereof).

In yet another aspect, methods for detecting human PDK1 and inhibiting human PDK1 activity, either in vitro or in vivo, using the PDK1 binding molecules of the invention (e.g. anti-PDK1 antibodies, or antigen-binding fragments thereof) are encompassed by the invention.

### I. Definitions

That the present invention may be more readily understood, select terms are defined below.

The terms “PDPK1,” “PDK1,” “human PDK1” or “hPDK1,” used interchangeably herein (PDK1 is also known as PDPK1), refer to 3-phosphoinositide-dependent protein kinase-1 (PDK1). Human PDPK1 (or human PDK1) is described in the Universal Protein Database (Uniprot database) under reference no. 015530. Exemplary amino acid sequences of human PDK1 are provided herein as SEQ ID NO: 16-20, as shown below in Table 1.

TABLE 1
Sequences of human PDK1, isoforms 1-5

| Sequence | Nucleic Acid or | |
|---|---|---|
| Identifier | Protein | Sequence |
| SEQ ID NO: 16 | Human PDK1 | MARTTSQLYDAVPIQSSVVLCSCPSPSMVRTQTESSTPPGIPGG |
| | isoform 1 protein | SRQGPAMDGTAAEPRP |
| | | GAGSLQHAQPPPQPRKKRPEDFKFGKILGEGSFSTVVLARELA |
| | | TSREYAIKILEKRHIIK |
| | | ENKVPYVTRERDVMSRLDHPFFVKLYFTFQDDEKLYFGLSYA |
| | | KNGELLKYIRKIGSFDET |
| | | CTRFYTAEIVSALEYLHGKGIIHRDLKPENILLNEDMHIQITDF |
| | | GTAKVLSPESKQARAN |
| | | SFVGTAQYVSPELLTEKSACKSSDLWALGCIIYQLVAGLPPFR |
| | | AGNEYLIFQKIIKLEYD |
| | | FPEKFFPKARDLVEKLLVLDATKRLGCEEMEGYGPLKAHPFF |
| | | ESVTWENLHQQTPPKLTA |
| | | YLPAMSEDDEDCYGNYDNLLSQFGCMQVSSSSSSHSLSASDT |
| | | GLPQRSGSNIEQYIHDLD |
| | | SNSFELDLQFSEDEKRLLLEKQAGGNPWHQFVENNLILKMGP |
| | | VDKRKGLFARRRQLLLTE |
| | | GPHLYYVDPVNKVLKGEIPWSQELRPEAKNFKTFFVHTPNRT |
| | | YYLMDPSGNAHKWCRKIQ |
| | | EVWRQRYQSHPDAAVQ |
| SEQ ID NO: 17 | Human PDK1 | MDGTAAEPRPGAGSLQHAQPPPQPRKKRPEDFKFGKILGEGS |
| | isoform 2 protein | FSTVVLARELATSREYAI |
| | | KILEKRHIIKENKVPYVTRERDVMSRLDHPFFVKLYFTFQDDE |
| | | KLYFGLSYAKNGELLKY |
| | | IRKIGSFDETCTRFYTAEIVSALEYLHGKGIIHRDLKPENILLNE |
| | | DMHIQITDFGTAKVL |
| | | SPESKQARANSFVGTAQYVSPELLTEKSACKSSDLWALGCIIY |
| | | QLVAGLPPFRAGNEYLI |
| | | FQKIIKLEYDFPEKFFPKARDLVEKLLVLDATKRLGCEEMEGY |
| | | GPLKAHPFFESVTWENL |
| | | HQQTPPKLTAYLPAMSEDDEDCYGNYDNLLSQFGCMQVSSS |
| | | SSSHSLSASDTGLPQRSGS |
| | | NIEQYIHDLDSNSFELDLQFSEDEKRLLLEKQAGGNPWHQFV |
| | | ENNLILKMGPVDKRKGLF |
| | | ARRRQLLLTEGPHLYYVDPVNKVLKGEIPWSQELRPEAKNFK |
| | | TFFVHTPNRTYYLMDPSG |
| | | NAHKWCRKIQEVWRQRYQSHPDAAVQ |
| SEQ ID NO: 18 | Human PDK1 | MARTTSQLYDAVPIQSSVVLCSCPSPSMVRTQTESSTPPGIPGG |
| | isoform 3 protein | SRQGPAMDGTAAEPRP |
| | | GAGSLQHAQPPPQPRKKRPEDFKFGKILGEGSFSTVVLARELA |
| | | TSREYAIKILEKRHIIK |
| | | ENKVPYVTRERDVMSRLDHPFFVKLYFTFQDDEKLYFGLSYA |
| | | KNGELLKYIRKIGSFDET |
| | | CTRFYTAEIVSALEYLHGKGIIHRDLKPENILLNEDMHIQITDF |
| | | GTAKVLSPESKQADLW |
| | | ALGCIIYQLVAGLPPFRAGNEYLIFQKIIKLEYDFPEKFFPKAR |
| | | DLVEKLLVLDATKRLG |
| | | CEEMEGYGPLKAHPFFESVTWENLHQQTPPKLTAYLPAMSED |
| | | DEDCYGNYDNLLSQFGCM |
| | | QVSSSSSSHSLSASDTGLPQRSGSNIEQYIHDLDSNSFELDLQF |
| | | SEDEKRLLLEKQAGGN |
| | | PWHQFVENNLILKMGPVDKRKGLFARRRQLLLTEGPHLYYV |
| | | DPVNKVLKGEIPWSQELRP |
| | | EAKNFKTFFVHTPNRTYYLMDPSGNAHKWCRKIQEVWRQRY |
| | | QSHPDAAVQ |
| SEQ ID NO: 19 | Human PDK1 | MARTTSQLYDAVPIQSSVVLCSCPSPSMVRTQTESSTPPGIPGG |
| | isoform 4 protein | SRQGPAMDGTAAEPRP |
| | | GAGSLQHAQPPPQPRKKRPEDFKFGKILGEGSFSTVVLARELA |
| | | TSREYATRANSFVGTAQ |
| | | YVSPELLTEKSACKSSDLWALGCIIYQLVAGLPPFRAGNEYLI |
| | | FQKIIKLEYDFPEKFFP |
| | | KARDLVEKLLVLDATKRLGCEEMEGYGPLKAHPFFESVTWE |
| | | NLHQQTPPKLTAYLPAMSE |
| | | DDEDCYGNYDNLLSQFGCMQVSSSSSSHSLSASDTGLPQRSG |
| | | SNIEQYIHDLDSNSFELD |
| | | LQFSEDEKRLLLEKQAGGNPWHQFVENNLILKMGPVDKRKG |
| | | LFARRRQLLLTEGPHLYYV |
| | | DPVNKVLKGEIPWSQELRPEAKNFKTFFVHTPNRTYYLMDPS |
| | | GNAHKWCRKIQEVWRQRY |
| | | QSHPDAAVQ |
| SEQ ID NO: 20 | Human PDK1 | MARTTSQLYDAVPIQSSVVLCSCPSPSMVRTQTESSTPPGIPGG |
| | isoform 5 protein | SRQGPAMDGTAAEPRP |
| | | GAGSLQHAQPPPQPRKKRPEDFKFGKILGEGSFSTVVLARELA |
| | | TSREYAIKILEKRHIIK |
| | | ENKVPYVTRERDVMSRLDHPFFVKLYFTFQDDEKLYFGLSYA |
| | | KNGELLKYIRKIGSFDET |
| | | CTRFYTAEIVSALEYLHGKGIIHRDLKPENILLNEDMHIQITDF |
| | | GTAKVLSPESKQARAN |
| | | SFVGTAQYVSPELLTEKSACKSSDLWALGCIIYQLVAGLPPFR |
| | | AGNEYLIFQKIIKLEYD |
| | | FPEKFFPKARDLVEKLLVLDATKRLGCEEMEGYGPLKAHPFF |
| | | ESVTWENLHQQTPPKLTA |
| | | YLPAMSEDDEDCYGNYDNLLSQFGCMQVSSSSSSHSLSASDT |
| | | GLPQRSGSNIEQYIHDLD |
| | | SNSFELDLQFSEDEKRLLLEKQAGGNPCLTGRII |

The term “extracellular PDK1”, as used herein, refers to a PDK1 protein in which at least a portion of the protein is outside of the cell, i.e., not intracellular. In one embodiment, the term “extracellular PDK1” refers to at least a portion of the PDK1 protein which is outside of the cell and is juxtaposed to or associated with the outer plasma membrane. In another embodiment, the term extracellular PDK1 refers to a portion of the PDK1 protein which is outside of the cell where the PDK1 protein is embedded in, associated with, or associated with the cell membrane. In another embodiment, the term extracellular PDK1 refers to a portion of the PDK1 protein which is outside of the an intact living cell and is not physically juxtaposed to the outer plasma membrane, i.e. is secreted.

The term “biological activity of PDK1”, as used herein, includes, in one embodiment, kinase activity, for example, serine/threonine kinase activity. In another embodiment, the biological activity of PDK1 includes the ability to initiate or enhance migration, invasiveness or metastasis of a cancer or tumor, including but not limited to breast cancer. In some embodiments, the biological activity of PDK1 includes phosphorylating protein kinase B (e.g. PKB/AKT1, PKB/AKT2, or PKB/AKT3), p70 ribosomal protein S6 kinase (RPS6KB1), p90 ribosomal protein S6 kinase (e.g. RPS6KA1, RPS6KA2 or RPS6KA3), cyclic AMP-dependent protein kinase (PRKACA), protein kinase C (e.g. PRKCD or PRKCZ), serum and glucocorticoid-inducible kinase (e.g. SGK1, SGK2 or SGK3), p21-activated kinase-1 (PAK1), and protein kinase PKN (e.g. PKN1 or PKN2). In some embodiments, the biological activity of PDK1 includes transduction of signals from insulin by providing the activating phosphorylation to PKB/AKT1. In some embodiments the activity of PDK1 includes negatively regulating TGF-beta-induced signaling by modulating the association of SMAD3 and/or SMAD7 with TGF-beta receptor, phosphorylating SMAD2, SMAD3, SMAD4 or SMAD7, preventing the nuclear translocation of SMAD3, SMAD4 and/or the translocation of SMAD7 from the nucleus to the cytoplasm in response to TGF-beta, activating PPARG transcriptional activity and promoting adipocyte differentiation, activating the NF-kappa-B pathway via phosphorylation of IKKB, regulating focal adhesions by angiotensin II, controlling proliferation, survival, and growth of developing pancreatic cells, regulating Ca2+ entry and/or Ca2+-activated K+ channels of mast cells, regulating the motility and/or chemotaxis of vascular endothelial cells (ECs), serving as a dual effector for cell survival and/or beta-adrenergic response in cardiac homeostasis, regulating thymocyte development by regulating the expression of key nutrient receptors on the surface of pre-T cells, mediating Notch-induced cell growth and/or proliferative responses, or providing negative feedback inhibition to toll-like receptor-mediated NF-kappa-B activation in macrophages. In some embodiments, the PDK1 exerts biological function through the phosphorylation of extracellular proteins, including soluble growth factors or cytokines, the extracellular domains of growth factor or cytokine receptors, or catalytic molecules such as matrix metalloproteases.

The terms “specific binding” or “specifically binding,” as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. In one embodiment, an anti-PDK1 antibody, or antigen-binding fragment thereof, specifically binds to an extracellular portion of a PDK1 protein.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter are lost in the presence of denaturing solvents.

The term “antibody,” as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding fragment” of an antibody (or simply “antibody fragment”), as used herein, refers to one or more fragments of an antibody that retain the ability to specifically bind to an antigen (e.g., PDK1 or extracellular PDK1). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see , Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding fragments are known in the art (Kontermann and Dubel eds., Antibody Engineering (2001) Springer-Verlag. New York. 790 pp. (ISBN 3-540-41354-5).

Still further, an antibody or antigen-binding fragment thereof may be part of a larger immunoadhesion molecules, formed by covalent or noncovalent association of the antibody or antibody fragment with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody fragments, such as Fab and F(ab′)₂ fragments, can be prepared from whole antibodies using conventional techniques, such as papain or pepsin digestion, respectively, of whole antibodies. Moreover, antibodies, antibody fragments and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds PDK1 is substantially free of antibodies that specifically bind antigens other than PDK1). An isolated antibody that specifically binds PDK1 may, however, have cross-reactivity to other antigens, such as PDK1 molecules from other species. Moreover, an isolated antibody may be substantially free of other cellular material and/or chemicals.

A “monoclonal antibody” as used herein refers to homogenous antibody population involved in the highly specific recognition and binding of a single antigenic determinant, or epitope. This is in contrast to polyclonal antibodies that typically include different antibodies directed against different antigenic determinants. Furthermore, “monoclonal antibody” refers to such antibodies made in any number of manners including but not limited to by hybridoma, phage selection, recombinant expression, and transgenic animals.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences. One embodiment provides fully human antibodies capable of binding human PDK1 which can be generated using techniques well known in the art, such as, but not limited to, using human Ig phage libraries such as those disclosed in Jermutus et al., PCT publication No. WO 2005/007699 A2.

The term “recombinant human antibody”, as used herein, is intended to include human antibodies that are prepared, expressed, created or isolated by recombinant means, i.e., wherein the antibody is expressed and purified in vitro. In one embodiment, a recombinant human antibody is expressed in a non-human host cell, e.g., a Chinese hamster ovary (CHO) cell or a mouse myeloma cell.

The term “labeled” as used herein, refers to a protein with a label incorporated that provides for the identification of the binding protein. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E.Coli; mammalian cell lines CHO, HEK 293 and COS; the insect cell line Sf9; and the fungal cell Saccharomyces cerevisiae.

The term “regulate” and “modulate” are used interchangeably, and, as used herein, refers to a change or an alteration in the activity or function of a molecule of interest (e.g., the biological activity of PDK1). Modulation may be an increase or a decrease in the magnitude of a certain activity or function of the molecule of interest (e.g. PDK1).

As used herein, the term “therapeutically effective amount” refers to the amount of a therapy, e.g., an anti-PDK1 antibody or antigen-binding fragment thereof, which is sufficient to reduce or ameliorate the severity and/or duration of a disorder, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g. , prophylactic or therapeutic agent).

As used herein, the term “a disorder in which PDK1 activity is detrimental” is intended to include diseases and other disorders in which the presence of PDK1 (e.g., extracellular PDK1) in a subject suffering from the disorder has been shown to be or is suspected of being either responsible for the pathophysiology of the disorder or a factor that contributes to a worsening of the disorder. Accordingly, a disorder in which PDK1 activity is detrimental is a disorder in which a decrease or inhibition of PDK1 activity is expected to alleviate the symptoms and/or progression of the disorder. Such disorders may be characterized by, for example, by expression of extracellular PDK1 which can be detected, for example, using an anti-PDK1 antibody or antibody fragment as described herein. A disorder in which PDK1 activity is detrimental includes cancer, such as metastatic cancer or non-resectable cancer, as described in detail herein. A disorder in which PDK1 activity is detrimental includes, in one embodiment, a cancer, e.g., metastatic or non-resectable cancer, that comprises or expresses extracellular PDK1.

The term “treating” includes the administration of the binding molecules, e.g., antibodies or agents of the present invention to prevent or delay the onset of the symptoms, complications, or biochemical indicia of a disease, or to alleviate the symptoms or arrest or inhibit further development of the disease, condition, or disorder (e.g., cancer). Treatment may be prophylactic (to prevent or delay the onset of the disease, or to prevent the manifestation of clinical or subclinical symptoms thereof) or therapeutic suppression or alleviation of symptoms after the manifestation of the disease. In one embodiment, the antibodies of the invention are used to treat cancer.

Except when noted, the terms “patient” or “subject” are used interchangeably and refer to mammals such as human patients and non-human primates, as well as experimental animals such as rabbits, rats, and mice, and other animals.

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells (including tumor cells or cancer cells), organs, tissues, including tumor tissue, bone marrow, lymph nodes and spleen.

As used herein, the terms “chimeric antigen receptor” or “CAR”, used interchangeably herein, refer to a fused protein comprising an extracellular domain capable of binding to an antigen (e.g., a tumor antigen), a transmembrane domain, and at least one intracellular signaling domain. The “extracellular domain capable of binding to an antigen” means any oligopeptide or polypeptide that can bind to a certain antigen, such as PDK1, particularly extracellular PDK1. In one embodiment, the transmembrane domain of the CAR is derived from a polypeptide different from the polypeptide from which the extracellular domain is derived.

An “immune effector cell,” as used herein, refers to any cell of the immune system that has one or more effector functions (e.g., cytotoxic cell killing activity, secretion of cytokines, induction of ADCC and/or CDC). In one embodiment, the immune effector cells used with the CARs as described herein are T lymphocytes, in particular cytotoxic T cells (CTLs; CD8+ T cells) and helper T cells (HTLs; CD4+ T cells). Other populations of T cells are also useful herein, for example naive T cells and memory T cells. As would be understood by the skilled person, other cells may also be used as immune effector cells with the CARs as described herein. In particular, immune effector cells also include NK cells, NKT cells, neutrophils, and macrophages Immune effector cells also include progenitors of effector cells wherein such progenitor cells can be induced to differentiate into an immune effector cells in vivo or in vitro. Thus, in this regard, immune effector cell includes progenitors of immune effectors cells such as hematopoietic stem cells (HSCs) contained within the CD34+ population of cells derived from cord blood, bone marrow or mobilized peripheral blood which upon administration in a subject differentiate into mature immune effector cells, or which can be induced in vitro to differentiate into mature immune effector cells.

### II. PDK1 Binding Molecules

PDK1 is a serine threonine kinase of the AGC family that plays an important role in Akt mediated signaling in cancer cell growth and proliferation. Additionally, PDK1 has been shown to act independently of the Akt pathway in human cancer (Vasudevan et al., 2009, Cancer Cell, 16(1): p. 21-32) and has been identified as a cancer target (Raimondi et al., 2011, Curr Med Chem 18(18): p. 2763-9). Furthermore, increasing evidence reveals that PDK1 plays an important role in breast cancer (Fyffe et al., 2013, Cancer Manag Res 5: p. 271-80). Over 70% of invasive breast carcinomas and 86% of high-grade metastasized breast tumors had moderate to high levels of PDK1 phosphorylation (Lin et al., 2005, Br J Cancer 93(12): 1372-81). PDK1 protein expression is elevated in most human breast cancers and 21% of tumors have PDK1 gene amplification that was associated with patient survival, implicating PDK1 alterations in oncogenic signaling in breast cancer (Maurer et al., 2009, Cancer Res 69(15): 6299-306). In a recent study, moderate or high expression of PDK1 protein was seen in 88% of breast cancer tumors (Arsenic, 2014, Diagn Pathol 9: 82). This did not correlate with PIK3CA mutation status, indicating a distinct role for PDK1 in breast cancer, separate from the PI3K/Akt pathway. Additionally, PDK1′s potential role in the EMT was exemplified by findings that PDK1 promotes invasion of the extracellular matrix and activation of matrix metalloproteinases that are positively correlated with metastasis (Xie et al., 2006, BMC Cancer 6: p. 77). Intracellular PDK1 in tumors can be inhibited by small molecule kinase inhibitors, but the discovery described herein that PDK1 is expressed on the outer plasma membrane of cancer cells establishes the novel paradigm that extracellular PDK1 can be specifically targeted, e.g., with monoclonal antibody-based therapies.

For example, binding of the human antibody 2B9 to the PDK1 protein is specific for cancer by virtue of its expression only on the surface of cancer cells. Furthermore, the limited binding of 2B9 to normal tissues suggests that the PDK1 epitope of 2B9 is masked in the cytoplasmic form of PDK1. Targeting extracellular PDK1 with binding molecules, such as 2B9, may therefore enable a novel therapeutic approach (e.g., a specific immunotherapy) for metastatic cancer. Importantly, no antibody specific for an extracellular domain of PDK1 has yet been described.

Thus, in one embodiment, the binding molecules of the invention, e.g., anti-PDK1 antibodies, or antigen-binding fragments thereof, bind to extracellular PDK1. In certain embodiments, the binding molecules, e.g. anti-PDK1 antibodies, or antigen binding fragments thereof, bind to a cell expressing extracellular PDK1. In further embodiments, the binding molecules of the invention, e.g., anti-PDK1 antibodies, or antigen-binding fragments thereof, bind to cancer cells expressing PDK1 on the surface of the cell, but do not bind to normal cells or tissue where PDK1 is expressed in the cytoplasm and nucleus of the cells.

In one aspect, the invention provides an antibody, or antigen binding fragment thereof, which binds to extracellular PDK1. In one embodiment, the present invention provides isolated human monoclonal anti-PDK1 antibodies, or antigen-binding fragments thereof, that bind to extracellular PDK1 with high affinity, and with a slow off rate. In one aspect, the extracellular PDK1 binding molecules, e.g., anti-PDK1 antibodies, and antigen-binding fragments thereof, of the invention bind to the extracellular portion of PDK1. In another aspect, the extracellular PDK1 binding molecules, e.g., anti-PDK1 antibodies, or antigen-binding fragments thereof, bind to cells comprising extracellular PDK1, and do not bind to a cell which does not express extracellular PDK1.

In another embodiment, an extracellular PDK1 binding molecule, e.g., anti-PDK1 antibody, or antigen-binding fragment thereof, of the invention, is capable of modulating one or more biological activity of PDK1. For example, binding of an extracellular PDK1 binding molecule, e.g., an antibody, or antigen-binding fragment thereof of the invention, to PDK1, e.g., the extracellular domain of PDK1 or a cell comprising extracellular PDK1, results in the modulation, e.g., decrease or inhibition, of one or more biological activities of PDK1.

In one embodiment, a PDK1 biological activity that can be modulated, e.g., decreased, includes, but is not limited to, kinase activity, for example, serine/threonine kinase activity. In another embodiment, an extracellular PDK1 binding molecule, e.g., anti-PDK1 antibody or antigen-binding fragment thereof, of the invention can modulate, e.g., decrease, the ability of PDK1 to initiate or enhance migration, invasion, and/or metastasis of a cancer, for example, breast cancer, cervical cancer or squamous cell carcinoma. In other embodiments of the foregoing aspects, the extracellular PDK1 binding molecule, e.g., PDK1 binding protein, e.g., antibody, or antigen binding fragment thereof, is capable of neutralizing PDK1.

Exemplary assays for assessing the activity of anti-PDK1 antibodies of the present invention include assays as set forth herein. For example, the assays may be used to determine whether an anti-PDK1 antibody inhibits PDK1 activity. Cell migration is an invasion assay that can be employed, as set forth herein. Inhibition of PDK1 kinase activity can also be assessed in vitro using kinase assays, (see, for example, the Promega PDK1 Kinase Enzyme System and the ADP-G1 Kinase Assay (Cat.# V9101, the protocols of which are publicly available on line at promega.com/resources/protocols/product-information-sheets/n/pdk1-kinase-enzyme-system-protocol/)). PDK1 kinase activity can further be measured in cells by testing whether PDK1 to phosphorylate AKT or other AGC group kinases in a Phosphoinositide 3-kinase dependent manner.

In one embodiment of the invention, an anti-PDK1 antibody, or antigen binding fragment thereof, comprises a light chain variable region comprising a CDR3 having an amino acid sequence of SEQ ID NO: 14 and/or a heavy chain variable region comprising a CDR3 having an amino acid sequence of SEQ ID NO:7. In certain embodiments, the anti-PDK1 antibody, or antigen binding fragment thereof, comprises a light chain variable region that comprises a CDR3 having the amino acid sequence of SEQ ID NO: 14, and a heavy chain variable region that comprises a CDR3 having the amino acid sequence of SEQ ID NO: 7.

In further embodiments of the invention, the heavy chain variable region of the anti-PDK1 antibody, or antigen binding fragment thereof, further comprises a CDR2 having an amino acid sequence of SEQ ID NO: 6. In other further embodiments, the light chain variable region of the anti-PDK1 antibody, or antigen binding fragment thereof, further comprises a CDR2 having an amino acid sequence set forth in SEQ ID NO: 13.

In other further embodiments of the invention, the heavy chain variable region of the anti-PDK1 antibody, or antigen binding fragment thereof, further comprises a CDR1 having an amino acid sequence of SEQ ID NO:5. In other further embodiments, the light chain variable region of the anti-PDK1 antibody, or antigen binding fragment thereof, further comprises a CDR1 having an amino acid sequence of SEQ ID NO: 12.

Accordingly, the present invention includes, in certain embodiments, an anti-PDK1 antibody, or antigen binding fragment thereof, comprising a heavy chain CDR1 comprising an amino acid sequence of SEQ ID NO:5, a heavy chain CDR2 comprising an amino acid sequence of SEQ ID NO:6, a heavy chain CDR3 comprising an amino acid sequence of SEQ ID NO:7, a light chain CDR1 comprising an amino acid sequence of SEQ ID NO:12, a light chain CDR2 comprising an amino acid sequence of SEQ ID NO:13, and a light chain CDR3 comprising an amino acid sequence of SEQ ID NO:14.

In one embodiment, the anti-PDK1 antibody, or antigen binding fragment thereof, comprises a heavy chain variable region having a CDR set (CDR1/CDR2/CDR3) as described in the amino acid sequence set forth in SEQ ID NO: 4. In one embodiment, the anti-PDK1 antibody, or antigen binding fragment thereof, comprises a light chain variable region having a CDR set (CDR1/CDR2/CDR3) as described in the amino acid sequence set forth in SEQ ID NO: 11.

The antibodies of the invention can be selected from any class of immunoglobulins, including IgM, IgG, IgD, IgA and IgE, and any isotype, including without limitation IgG 1, IgG2, IgG3 and IgG4. The antibody may comprise sequences from more than one class or isotype, and particular constant domains may be selected to optimize desired effector functions using techniques well-known in the art. In one embodiment, the anti-PDK1 antibody, or antigen binding fragment thereof, is an IgG isotype, for example IgG1-K.

In a particular embodiment, the anti-PDK1 antibody, or antigen binding fragment thereof, comprises a heavy chain variable region comprising the amino acid sequence set forth in SEQ ID NO: 4. In a related embodiment, the heavy chain variable region is encoded by a nucleic acid comprising the sequence set forth in SEQ ID NO: 3. In another embodiment, the anti-PDK1 antibody, or antigen binding fragment thereof, comprises a light chain variable region comprising the amino acid sequence set forth in SEQ ID NO: 11. In a related embodiment, the light chain variable region is encoded by a nucleic acid comprising the sequence set forth in SEQ ID NO: 10.

In another embodiment of the invention, the anti-PDK1 antibody, or antigen binding fragment thereof, comprises a heavy chain variable domain comprising an amino acid sequence set forth in SEQ ID NO: 4 and a light chain variable domain comprising an amino acid sequence set forth in SEQ ID NO: 11. In a further embodiment, the anti-PDK1 antibody, or antigen binding fragment thereof, comprises a heavy chain variable region comprising an amino acid sequence set forth in SEQ ID NO: 4, or a sequence having at least 90%, 91%, 92%, 93%, 94% 95%, 96%, 97%, 98%, or 99% identity to SEQ ID NO: 4, and/or a light chain comprising an amino acid sequence set forth in SEQ ID NO: 11, or a sequence having at least 90%, 91%, 92%, 93%, 94% 95%, 96%, 97%, 98%, or 99% identity to SEQ ID NO: 11.

In one embodiment of the invention, the heavy chain of the anti-PDK1 antibody, or antigen binding fragment thereof, comprises an amino acid sequence set forth in SEQ ID NO: 2, and a light chain comprising an amino acid sequence set forth in SEQ ID NO: 9. In a further embodiment, the anti-PDK1 antibody, or antigen binding fragment thereof, comprises a heavy chain comprising an amino acid sequence set forth in SEQ ID NO: 2, or a sequence having at least 90%, 91%, 92%, 93%, 94% 95%, 96%, 97%, 98%, or 99% identity to SEQ ID NO: 2, and/or a light chain comprising an amino acid sequence set forth in SEQ ID NO: 9, or a sequence having at least 90%, 91%, 92%, 93%, 94% 95%, 96%, 97%, 98%, or 99% identity to SEQ ID NO: 9.

In one embodiment, the antibody of the invention is a recombinant human antibody. In one embodiment, the antibody is a bispecific antibody.

According to certain embodiments of the present invention, the anti-PDK1 antibody, or antigen binding fragment thereof, comprises a heavy chain immunoglobulin constant domain of a human IgM constant domain, a human IgG1 constant domain, a human IgG2 constant domain, a human IgG3 constant domain, a human IgG4 constant domain, a human IgA constant domain, or a human IgE constant domain.

In one non-limiting embodiment, an anti-PDK1 antibody, or antigen-binding fragment thereof is the 2B9 monoclonal antibody, described herein in the Examples and the sequences described in the sequence summary. The hybridoma that produces the anti-PDK1 antibody 2B9 was deposited under the Budapest Treaty on ______, with the American Type Culture Collection, 10801 University Boulevard, Manassas, Va. 20110-2209 (ATCC Deposit No. ______).

In one embodiment of the invention, the monoclonal anti-PDK1 antibody comprises a light chain variable domain comprising the CDRs of antibody 2B9 (ATCC Deposit No. ______) and a heavy chain variable domain comprising the CDRs of antibody 2B9. In one embodiment of the invention, the monoclonal anti-PDK1 antibody comprises a light chain variable domain of antibody 2B9 (ATCC Deposit No. ______) and a heavy chain variable domain of antibody 2B9.

In one embodiment, the invention provides a bispecific T cell engager (BITE) which has specificity for PDK1 (as a tumor antigen associated with the surface of a tumor cell) and CD3. More specifically, a BITE of the invention is designed such that one domain of the bispecific protein targets an antigen on the surface of a cancer cell, e.g., PDK1, whereas the other domain is designed to engage CD3 on the surface of a CTL. Thus, the bispecific T cell engager creates a bridge between the CTL and the malignant cell and helps to facilitate targeted cell lysis. See Nagorsen et al. (2000) Cancer Res. 60:4850. Baeuerle and Reinhardt (Cancer Res 2009; 69: (12). Jun. 15, 2009) review the principles of BITE antibodies and discuss the contribution of BITE antibodies to the immunotherapy of cancer.

In one embodiment of the invention, antibody fragments that bind PDK1 comprise the antigen binding regions described herein. Antibody fragments which recognize PDK1, particularly extracellular PDK1, can be generated by known techniques. Antigen binding portions (or fragments) of an antibody include the following: F(ab)₂, Fab′, Fab, Fv, and scFv.

In certain embodiments, the antigen binding fragment of the invention comprises a Single-Chain Fragment Variable (scFv). An Fv fragment is the smallest unit of immunoglobulin molecule with function in antigen-binding. An scFv is an antibody fragment obtained by ligating the C-terminus of one chain of Fv consisting of an H chain variable region (VH) and an L chain variable region (VL) to the N-terminus of the other chain thereof, using a suitable peptide linker, so as to form a single chain. For example, the linker (GGGGS)₃ (SEQ ID NO:21) may be used as, for example, a peptide linker and has high flexibility. For instance, DNA encoding the H chain variable region and L chain variable region of the above-described antibody and DNA encoding a peptide linker are used to construct DNA encoding a scFv antibody, and the thus constructed DNA is then incorporated into a suitable vector. Thereafter, scFv can be prepared from a transformant obtained by transformation with the aforementioned vector.

Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain. An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

In certain embodiments, the ScFv comprises the amino acid sequence of SEQ ID NO:15.

Anti-PDK1 antibodies can be identified using a wide variety of techniques known in the art, including the use of hybridoma, recombinant, and phage display technologies, or a combination thereof. For example, monoclonal antibodies can be produced using hybridoma techniques including those known in the art and taught, for example, in Harlow et al. , Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties).

Methods for producing and screening for specific antibodies using hybridoma technology are routine and well known in the art. In one embodiment, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of interest (PDK1) with myeloma cells and then screening the hybridomas resulting from the fusion for hybridoma clones that secrete an antibody able to bind a polypeptide of the invention. See, e.g., Harlow and Lane, supra.

In another embodiment of the instant invention, anti-PDK1 antibodies are produced by immunizing a non-human animal comprising some, or all, of the human immunoglobulin locus with a PDK1 antigen. In a preferred embodiment, the non-human animal is a XENOMOUSE transgenic mouse, an engineered mouse strain that comprises large fragments of the human immunoglobulin loci and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The XENOMOUSE transgenic mouse produces an adult-like human repertoire of fully human antibodies, and generates antigen-specific human Mabs. The XENOMOUSE transgenic mouse contains approximately 80% of the human antibody repertoire through introduction of megabase sized, germline configuration YAC fragments of the human heavy chain loci and x light chain loci. See Mendez et al., Nature Genetics 15:146-156 (1997), Green and Jakobovits J. Exp. Med. 188:483-495 (1998), the disclosures of which are hereby incorporated by reference.

In vitro methods also can be used to make anti-PDK1 antibodies of the invention, wherein an antibody library is screened to identify an antibody having the desired binding specificity. Methods for such screening of recombinant antibody libraries are well known in the art and include methods described in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, US patent application publication 20030186374, and PCT Publication No. WO 97/29131, the contents of each of which are incorporated herein by reference.

The recombinant antibody library may be from a subject immunized with PDK1, or preferably a portion of PDK1, such as the extracellular domain. Alternatively, the recombinant antibody library may be from a naïve subject, i.e., one who has not been immunized with PDK1, such as a human antibody library from a human subject who has not been immunized with human PDK1. Antibodies of the invention may be selected by screening the recombinant antibody library with the peptide comprising human PDK1 to thereby select those antibodies that recognize PDK1. Methods for conducting such screening and selection are well known in the art. To select antibodies of the invention having particular binding affinities for hPDK1, such as those that dissociate from human PDK1 with a particular koff rate constant, the art-known method of surface plasmon resonance can be used to select antibodies having the desired koff rate constant.

Anti-PDK1 antibodies can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780, 225; 5,658,727; 5,733,743 and 5,969,108; each of which is incorporated herein by reference in its entirety.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

In another approach the antibodies of the present invention can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present invention include those disclosed in Wittrup et al. (U.S. Pat. No. 6,699,658) incorporated herein by reference.

Antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the invention in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In one embodiment, the antibody of the invention is produced using a non-human mammalian host cell, such as, but not limited to, a CHO cell, a NSO cell, or an Sp2 cell. In one embodiment, a recombinant human antibody of the invention is produced using a non-human mammalian host cell, such as, but not limited to, a CHO cell, a NSO cell, or a Sp2 cell.

When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In various embodiments of the foregoing aspects, the extracellular PDK1 binding protein, e.g., antibody, or antigen binding portion thereof, of the invention is capable of modulating one or more biological activities of PDK1. In other embodiments of the foregoing aspects, the extracellular PDK1 binding protein, e.g., antibody, or antigen binding fragment thereof, is capable of inhibiting one or more biological activities of PDK1. In other embodiments of the foregoing aspects, the extracellular PDK1 binding protein is capable of inhibiting one or more biological activities of extracellular PDK1.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

### III. Screening Assays for PDK1 Binding Molecules

In certain aspects, the invention provides methods (also referred to herein as “screening assays”) for identifying binding molecules that bind to extracellular PDK1. The invention also provides methods for identifying binding molecules which bind to a cell expressing extracellular PDK1 and do not bind to a cell which does not express extracellular PDK1. Such assays typically comprise a reaction between extracellular PDK1 and one or more assay components. The other components may be either the test compound itself, or a combination of test compounds and a natural binding partner of extracellular PDK1 (e.g., the antibody 2B9), e.g., for use in carrying out competition assays. Compounds identified via assays such as those described herein may be useful, for example, for diagnosing or treating cancer, e.g., metastatic or non-resectable cancer and/or a cancer characterized by expression of extracellular PDK1. Compounds identified for binding to extracellular PDK1 are preferably further tested for activity useful in the treatment of cancer, e.g., cancer characterized by expression of extracellular PDK1, such as the ability to inhibit migration, invasion or metastasis of tumor cells, inhibiting tumor cell growth, etc.

The test compounds used in the screening assays of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

The invention provides assays for screening candidate or test molecules which bind to extracellular PDK1 or biologically active portions thereof. Determining the ability of the test molecule to directly bind to extracellular PDK1 can be accomplished, for example, by any method known in the art. The screening methods of the invention comprise contacting a cell, e.g., a cell expressing extracellular PDK1 (e.g., a breast cancer cell expressing extracellular PDK1) with a test molecule and determining the ability of the test molecule to bind to said cell and not bind to a cell which does not express extracellular PDK1. The screening methods may comprise determining the ability of the test molecule to modulate an activity of PDK1, preferably an activity of extracellular PDK1. The activity of PDK1, preferably activity of extracellular PDK1, can be determined using any methods known in the art, such as those described herein. In one embodiment, the activity of PDK1 is kinase activity. In on embodiment, the screening methods may comprise determining the ability of the test molecule to inhibit migration of a cancer cell expressing extracellular PDK1. Exemplary assays for measuring migration of cancer cells are described herein and additional assays are well known in the art.

This invention further pertains to novel agents identified by the above-described screening assays. Accordingly, it is within the scope of this invention to further use an agent identified as described herein in an appropriate animal model. For example, an agent capable of modulating the activity of extracellular PDK1 identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment as described herein.

### IV. PDK1 Inhibitors

In certain aspects, the invention relates to a method of treating a cancer in a subject in need thereof comprising obtaining a sample from a subject having cancer, testing the sample for expression of PDK1, and administering to the subject a therapeutically effective amount of a PDK1 inhibitor.

In certain embodiments, the PDK1 inhibitor is a small molecule inhibitor. As used herein, a “small molecule” inhibitor is an inhibitor molecule that has a molecular weight of less than 1000 Da, preferably less than 750 Da, or preferably less than 500 Da. In certain embodiments, a small molecule inhibitor does not include a nucleic acid molecule. In certain embodiments, a small molecule inhibitor does not include a peptide more than three amino acids in length.

PDK1 inhibitors include for example BX912 (CAS Accession Number: 702674-56-4) and BX795 (CAS Accession Number: 702675-74-9). Other PDK1 inhibitors are known in the art and are described, for example, in Feldman et al., 2005, J. Biol. Chem. 280(20): 19867-19874 and Peifer et al., 2008, ChemMedChem. 3:10, each of which is expressly incorporated by reference herein in its entirety.

### V. Anti-PDK1 Chimeric Antigen Recepor (CAR)

Chimeric antigen receptors (CARs) redirect T cell specificity toward antibody-recognized antigens expressed on the surface of cancer cells, while T cell receptors (TCRs) extend the range of targets to include intracellular tumor antigens. CAR redirected T cells specific for the B cell differentiation antigen CD19 have shown dramatic efficacy in the treatment of B cell malignancies, while TCR-redirected T cells have shown benefits in patients suffering from solid cancer. Stauss et al. describe strategies to modify therapeutic CARs and TCRs, for use in the treatment of cancer, for example, to enhance the antigen-specific effector function and limit toxicity of engineered T cells (Current Opinion in Pharmacology 2015, 24:113-118).

One aspect of the invention includes a chimeric antigen receptor (CAR) which is specific for the tumor antigen PDK1 as the antigen is expressed on the surface of tumor cells. In on embodiment of the present invention, a CAR as described herein comprises an extracellular target-specific binding domain, a transmembrane domain, an intracellular signaling domain, such as a signaling domain derived from CD3zeta or FcRgamma, and optionally, one or more co-stimulatory signaling domains derived from a co-stimulatory molecule, such as, but not limited to, CD28, CD137, CD134 or CD278. In one embodiment, the CAR includes a hinge or spacer region inbetween the extracellular binding domain and the transmembrane domain.

As used herein, the binding domain or the extracellular domain of the CAR, provides the CAR with the ability to bind to the target antigen of interest, i.e., extracellular PDK1. A binding domain can be any protein, polypeptide, oligopeptide, or peptide that possesses the ability to specifically recognize and bind to a biological molecule (e.g., a cell surface receptor or tumor protein, or a component thereof). A binding domain includes any naturally occurring, synthetic, semi-synthetic, or recombinantly produced binding partner for a biological molecule of interest. For example, and as further described herein, a binding domain may be antibody light chain and heavy chain variable regions, or the light and heavy chain variable regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE analysis). The target may be any antigen of clinical interest against which it would be desirable to trigger an effector immune response that results in tumor killing. In one embodiment, the target antigen of the binding domain of the chimeric antigen receptor is PDK1.

Illustrative binding domains include antigen binding fragments of an antibody, such as scFv, scTCR, extracellular domains of receptors, ligands for cell surface molecules/receptors, or receptor binding domains thereof, and tumor binding proteins. In certain embodiments, the antigen binding domains included in a CAR of the invention can be a variable region (Fv), a CDR, a Fab, an scFv, a VH, a VL, a domain antibody variant (dAb), a camelid antibody (VHH), a fibronectin 3 domain variant, an ankyrin repeat variant and other antigen-specific binding domain derived from other protein scaffolds.

In one embodiment, the binding domain of the CAR is an anti-PDK1 single chain antibody (scFv), and may be a murine, human or humanized scFv. Single chain antibodies may be cloned form the V region genes of a hybridoma specific for a desired target. The production of such hybridomas has become routine. A technique which can be used for cloning the variable region heavy chain (VH) and variable region light chain (VL) has been described, for example, in Orlandi et al., PNAS, 1989; 86: 3833-3837. Thus, in certain embodiments, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In certain embodiments, the CARs of the present invention may comprise linker residues between the various domains, added for appropriate spacing and conformation of the molecule. For example, in one embodiment, there may be a linker between the binding domain VH or VL which may be between 1-10 amino acids long. In other embodiments, the linker between any of the domains of the chimeric antigen receptor may be between 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long. Ranges including the numbers described herein are also included herein, e.g., a linker 10-30 amino acids long.

In certain embodiments, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

The “transmembrane,” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In one embodiment, the transmembrane domain is the transmembrane domain of CD8. In certain embodiments, the transmembrane domain is synthetic in which case it would comprise predominantly hydrophobic residues such as leucine and valine.

The “intracellular signaling domain,” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Primary cytoplasmic signaling sequences regulate primary activation of the T cell receptor complex either an inhibitory way, or in an inhibitory way. Primary cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular used in the invention include those derived from TCRzeta, FcRgamma, FcR.beta., CD3.gamma., CD3.delta., CD3.epsilon., CD5, CD22, CD79a, CD79b and CD66d. In certain particular embodiments, the intracellular signaling domain of the anti-PDK1 CARs described herein are derived from CD3zeta or FcRgamma.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains may enhance the efficacy and expansion of T cells expressing CAR receptors. The intracellular signaling and co-stimulatory signaling domains may be linked in any order in tandem to the carboxyl terminus of the transmembrane domain.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant co-stimulatory signal. Other CARs containing a binding domain, a hinge, a transmembrane and the signaling domain derived from CD3zeta or FcRgamma together with one or more co-stimulatory signaling domains (e.g., intracellular co-stimulatory domains derived from CD28, CD137, CD134 and CD278) has been shown to more effectively direct antitumor activity as well as increased cytokine secretion, lytic activity, survival and proliferation in CAR expressing T cells in vitro, in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

In one embodiment, the PDK1 CAR proteins may comprise (a) an anti-PDK1 scFv as a binding domain (e.g., an scFv having binding regions (e.g., CDRs or variable domains) from antibody 2B9) (b) a hinge region derived from human CD8alpha, (c) a human CD8alpha transmembrane domain, and (d) a human T cell receptor CD3 zeta chain (CD3) intracellular signaling domain, and optionally one or more co-stimulatory signaling domains derived from CD28, CD137, CD134, and CD278. In one embodiment, the different protein domains are arranged from amino to carboxyl terminus in the following order: binding domain, hinge region and transmembrane domain. The intracellular signaling domain and optional co-stimulatory signaling domains are linked to the transmembrane carboxy terminus in any order in tandem to form a single chain chimeric polypeptide. The nucleic acid construct encoding a PDK1 CAR is a chimeric nucleic acid comprising a nucleic acid sequence of different coding sequences, for example: the coding sequences of a human anti-PDK1 scFv, a human CD8alpha-hinge, a human CD28 transmembrane domain and a CD3zeta intracellular signaling domain.

In certain embodiments, the polynucleotide encoding the CAR described herein is inserted into a vector. The term “vector” as used herein refers to a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra. An expression vector may comprise additional elements, for example, the expression vector may have two replication systems, thus allowing it to be maintained in two organisms, for example in human cells for expression and in a prokaryotic host for cloning and amplification.

The expression vector should have the necessary 5′ upstream and 3′ downstream regulatory elements such as promoter sequences such as CMV, PGK and EF1 .alpha. promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector can have additional sequence such as 6x-histidine, c-Myc, and FLAG tags which are incorporated into the expressed CARs disclosed herein. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector sometimes also is engineered for replication and/or expression functionality (e.g., transcription and translation) in a particular cell type, cell location, or tissue type. Expression vectors sometimes include a selectable marker for maintenance of the vector in the host or recipient cell.

Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the chimeric proteins disclosed herein can be ligated into such expression vectors for the expression of the chimeric protein in mammalian cells.

In certain embodiments, the nucleic acids encoding the CAR of the present invention are provided in a viral vectors. A viral vector can be those derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for a scFvPDK1-CD3-.zeta. and the various chimeric proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the viral vector containing the coding sequence for a CAR described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). Retroviruses” of the invention also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immnodeficiency virus (EIV), and other classes of retroviruses. A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Preparation of the recombinant lentivirus can be achieved using the methods according to Dull et al. and Zufferey et al. (Dull et al., J. Virol., 1998; 72: 8463-8471 and Zufferey et al., J. Virol. 1998; 72:9873-9880).

Retroviral vectors for use in the present invention can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of the PDK1 CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CAR of the present invention are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or a may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding an anti-PDK1 CAR carried by a retroviral vector can be transduced into a cell through infection and pro virus integration.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In particular, the CAR of the present invention is introduced and expressed in immune effector cells so as to redirect their specificity to a target antigen of interest, e.g., PDK1.

The present invention provides methods for making the immune effector cells which express the CAR as described herein. In one embodiment, the method comprises transfecting or transducing immune effector cells isolated from an individual such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source of cells may be obtained from a subject. In particular, the immune effector cells for use with the CARs as described herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium in may lack magnesium or may lack many if not all divalent cations. As would be appreciated by those of ordinary skill in the art, a washing step may be accomplished by methods known to those in the art, such as by using a semiautomated flowthrough centrifuge. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present invention.

PBMC may be used directly for genetic modification with the CARs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion. CD8+ cells can be obtained by using standard methods. In some embodiments, CD8+ cells are further sorted into naive, central memory, and effector cells by identifying cell surface antigens that are associated with each of those types of CD8+ cells. In embodiments, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some embodiments, the expression of phenotypic markers of central memory TCM include CD45RO, CD62L, CCR7, CD28, CD3, and CD127 and are negative for granzyme B. In some embodiments, central memory T cells are CD45RO+, CD62L+, CD8+ T cells. In some embodiments, effector T cells are negative for CD62L, CCR7, CD28, and CD127, and positive for granzyme B and perforin. In some embodiments, naive CD8+ T lymphocytes are characterized by the expression of phenotypic markers of naive T cells including CD62L, CCR7, CD28, CD3, CD 127, and CD45RA.

In certain embodiments, CD4+ T cells are further sorted into subpopulations. For example, CD4+ T helper cells can be sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+CD4+ T cell. In some embodiments, central memory CD4+ cells are CD62L positive and CD45RO positive. In some embodiments, effector CD4+ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In another embodiment, the immune effector cells, such as T cells, are genetically modified with the chimeric antigen receptors described herein (e.g., transduced with a viral vector comprising a nucleic acid encoding a CAR) and then are activated and expanded in vitro. Methods for activating and expanding T cells are known in the art and are described, for example, in U.S. Pat. No. 6,905,874; U.S. Pat. No. 6,867,041; U.S. Pat. No. 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In other embodiments, the T cells may be activated and stimulated to proliferate with feeder cells and appropriate antibodies and cytokines using methods such as those described in U.S. Pat. No. 6,040,177; U.S. Pat. No. 5,827,642; and WO2012129514.

The invention provides a population of modified immune effector cells for the treatment of cancer, the modified immune effector cells comprising a PDK1 CAR as disclosed herein.

CAR-expressing immune effector cells prepared as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

A treatment-effective amount of cells in the composition is at least 2 cells (for example, at least 1 CD8+ central memory T cell and at least 1 CD4+ helper T cell subset) or is more typically greater than 10² cells, and up to 10⁶, up to and including 10⁸ or 10⁹ cells and can be more than 10¹⁰ cells. The number of cells will depend upon the ultimate use for which the composition is intended as will the type of cells included therein.

The cells may be autologous or heterologous to the patient undergoing therapy. If desired, the treatment may also include administration of mitogens (e.g., PHA) or lymphokines, cytokines, and/or chemokines (e.g., IFN-.gamma., IL-2, IL-12, TNF-alpha, IL-18, and TNF-beta, GM-CSF, IL-4, IL-13, Flt3-L, RANTES, MIP1.alpha., etc.) as described herein to enhance induction of the immune response.

The CAR expressing immune effector cell populations of the present invention may be administered either alone, or as a pharmaceutical composition in combination with diluents and/or with other components such as IL-2 or other cytokines or cell populations. Briefly, pharmaceutical compositions of the present invention may comprise a CAR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

The anti-tumor immune response induced in a subject by administering CAR expressing T cells described herein using the methods described herein, or other methods known in the art, may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present invention, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

Thus the present invention provides for methods of treating an individual diagnosed with or suspected of having, or at risk of developing, a PDK1-expressing cancer, comprising administering the individual a therapeutically effective amount of the CAR-expressing immune effector cells as described herein.

In one embodiment, the invention provides a method of treating a subject diagnosed with a PDK1-expressing cancer comprising removing immune effector cells from a subject diagnosed with a PDK1-expressing cancer, genetically modifying said immune effector cells with a vector comprising a nucleic acid encoding a chimeric antigen receptor of the instant invention, thereby producing a population of modified immune effector cells, and administering the population of modified immune effector cells to the same subject. In one embodiment, the immune effector cells comprise T cells.

The methods for administering the cell compositions described herein includes any method which is effective to result in reintroduction of ex vivo genetically modified immune effector cells that either directly express a CAR of the invention in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct in accordance with the invention and returning the transduced cells into the subject.

### VI. Therapeutic Uses of PDK1 Binding Molecules

The extracellular PDK1-binding molecules, e.g., antibodies and antibody fragments, of the invention preferably are capable of modulating human PDK1 activity (e.g. extracellular PDK1 activity) both in vitro and in vivo. Accordingly, such binding molecules, e.g., antibodies and antibody fragments of the invention can be used to modulate, e.g. inhibit, PDK1 biological activity, e.g., in a cell culture containing PDK1, or in vivo in human subjects, or in other mammalian subjects having PDK1 with which an antibody of the invention binds.

Accordingly, in one embodiment, the invention provides a method for modulating, e.g., decreasing or inhibiting PDK1 (e.g. extracellular PDK1) biological activity comprising contacting PDK1 (e.g. extracellular PDK1) with an extracellular PDK1-binding molecule, e.g., an antibody or antibody fragment, of the invention such that PDK1 biological activity is decreased. For example, in a cell culture containing, or suspected of containing PDK1 (e.g. extracellular PDK1), an extracellular PDK1-binding molecule, e.g., antibody or antibody fragment of the invention can be added to the culture medium to inhibit PDK1 biological activity in the culture.

In another embodiment, the invention provides a method for decreasing one or more PDK1 (e.g. extracellular PDK1) biological activities in a subject, advantageously in a subject suffering from a disease or disorder in which PDK1 (e.g. extracellular PDK1) activity is detrimental, such as cancer, thereby treating the disease or disorder. The invention provides methods for decreasing one or more PDK1 (e.g. extracellular PDK1) biological activities in a subject suffering from such a disease or disorder, which method comprises administering to the subject an extracellular PDK1-binding molecule, e.g., antibody or antibody fragment of the invention such that PDK1 (e.g. extracellular PDK1) activity in the subject is decreased and/or the disease or disorder is treated in the subject. Preferably, the PDK1 is human PDK1, and the subject is a human subject. Alternatively, the subject can be a mammal expressing a PDK1 to which an antibody of the invention is capable of binding. Still further the subject can be a mammal into which PDK1 has been introduced (e.g., by administration of PDK1 or by expression of a PDK1 transgene). An antibody of the invention can be administered to a non-human mammal expressing a PDK1 with which the antibody is capable of binding for veterinary purposes or as an animal model of human disease. Regarding the latter, such animal models may be useful for evaluating the therapeutic efficacy of antibodies of the invention (e.g., testing of dosages and time courses of administration).

In one embodiment, a disorder in which PDK1 activity is detrimental is a cancer in which the cancer is characterized as comprising extracellular PDK1 or expression of extracellular PDK1. The invention provides methods where cancer in a subject is treated by selecting a subject having cancer characterized by expression of extracellular PDK1, and administering to the subject a therapeutically effective amount of an extracellular PDK1-binding molecule, e.g., antibody, or antigen binding fragment thereof, of the invention, thereby treating the cancer in the subject.

In another aspect of the invention, a subject having cancer can be treated wherein the subject is identified by obtaining a sample from a subject having cancer, and testing the sample for expression of extracellular PDK1 using an extracellular PDK1-binding molecule, e.g., antibody or antibody fragment of the invention. If extracellular PDK1 is identified in the sample, the subject can be treated by administering a therapeutically effective amount of the extracellular PDK1-binding molecule, e.g., antibody, or antigen binding fragment thereof of the invention.

Non-limiting examples of disorders that can be treated with the antibodies of the invention, for example, 2B9, variants thereof, or antigen binding fragments thereof, include, for example, but are not limited to, a variety of cancers including, melanoma, squamous cell carcinoma, lymphoma, breast cancer, ovarian cancer, cervical cancer, renal carcinoma, gastrointestinal cancer, colon cancer, lung cancer, pancreatic cancer, endometrial cancer and prostate cancer. In particular embodiments, the cancer is breast cancer, renal cancer, prostate cancer, ovarian cancer, endometrial cancer or lung cancer. In a particular embodiment, the cancer is breast cancer, cervical cancer, or squamous cell carcinoma. In one embodiment, the cancer is acute myeloid leukemia (AML). In one embodiment, the cancer is a solid tumor. In one embodiment, the cancer is a hematopoietic cancer.

In particular embodiments, the extracellular PDK1-binding molecules, e.g., anti-PDK1 antibodies or antibody fragments can be administered alone or with another anti-neoplastic agent which acts in conjunction with or synergistically with the antibody to treat the disease associated with PDK1 activity, e.g., cancer. The term “anti-neoplastic agent” refers to a composition useful in treating cancer comprising at least one active therapeutic agent, e.g., “anti-cancer agent.” Examples of therapeutic agents (anti-cancer agents) include, but are limited to, e.g., chemotherapeutic agents, growth inhibitory agents, cytotoxic agents, agents used in radiation therapy, anti-angiogenic agents, apoptotic agents, anti-tubulin agents, small molecules, anti-cancer antibodies, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva), platelet derived growth factor inhibitors (e.g., Gleevec (Imatinib Mesylate)), a COX-2 inhibitor (e.g., celecoxib), interferons, cytokines, antagonists (e.g., neutralizing antibodies) that bind to one or more of the following targets: ErbB2, ErbB3, ErbB4, PDGFR-beta, BlyS, APRIL, BCMA or VEGF receptor(s), TRAIL/Apo2, and other bioactive and organic chemical agents, etc.

Examples of other anti-cancer agents include, but are not limited to, Panorex (Glaxo-Welcome), Rituxan (IDEC/Genentech/Hoffman la Roche), Mylotarg (Wyeth), Campath (Millennium), Zevalin (IDEC and Schering AG), Bexxar (Corixa/GSK), Erbitux (Imclone/BMS), Avastin (Genentech) and Herceptin (Genentech/Hoffman la Roche). Other anti-cancer agents include, but are not limited to, those disclosed in U.S. Pat. No. 7,598,028, U.S. Pat. No. 8,101,177, and International Publication No. WO2008/100624, the contents of which are hereby incorporated by reference.

The term “cytotoxic agent” or “cytotoxin” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶)chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include is a chemical compound useful in the treatment of cancer. Examples of chemotherapeutic agents include alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, Ill.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE vinorelbine;

novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva)) and VEGF-A that reduce cell proliferation and pharmaceutically acceptable salts, acids or derivatives of any of the above.

Also included are anti-hormonal agents that act to regulate or inhibit hormone action on tumors such as anti-estrogens and selective estrogen receptor modulators (SERMs), including, for example, tamoxifen (including NOLVADEX tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE megestrol acetate, AROMASIN exemestane, formestanie, fadrozole, RIVISOR vorozole, FEMARA letrozole, and ARIMIDEX anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME ribozyme) and a HER2 expression inhibitor; vaccines such as gene therapy vaccines, for example, ALLOVECTIN vaccine, LEUVECTIN vaccine, and VAXID vaccine; PROLEUKIN rIL-2; LURTOTECAN topoisomerase 1 inhibitor; ABARELIX rmRH; Vinorelbine and Esperamicins (see U.S. Pat. No. 4,675,187), and pharmaceutically acceptable salts, acids or derivatives of any of the above.

The term “prodrug” refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, .beta.-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

An “anti-angiogenic agent” or “angiogenesis inhibitor” refers to a small molecular weight substance, an polynucleotide, an polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or to the VEGF-A receptor (e.g., KDR receptor or Flt-1 receptor), anti-PDGFR inhibitors such as Gleevec (Imatinib Mesylate). Anti-angiogenesis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) (e.g., Table 2 listing known antiangiogenic factors); and, Sato Int. J. Clin. Oncol., 8:200-206 (2003) (e.g., Table 1 lists anti-angiogenic agents used in clinical trials).

One or more anti-cancer agents may be administered either simultaneously or before or after administration of an extracellular PDK1-binding molecule, e.g., antibody or antigen binding fragment thereof of the present invention.

Further examples of preferred additional therapeutic agents that can be co-administered and/or formulated with one or more extracellular PDK1-binding molecules, e.g., anti-PDK1 antibodies or fragments thereof, include, but are not limited to, one or more of: inhaled steroids; beta-agonists, e.g., short-acting or long-acting beta-agonists; antagonists of leukotrienes or leukotriene receptors; combination drugs such as ADVAIR; IgE inhibitors, e.g., anti-IgE antibodies (e.g., XOLAIR); phosphodiesterase inhibitors (e.g., PDE4 inhibitors); xanthines; anticholinergic drugs; mast cell-stabilizing agents such as cromolyn; IL-4 inhibitors; IL-5 inhibitors; eotaxin/CCR3 inhibitors; antagonists of histamine or its receptors including H1, H2, H3, and H4, and antagonists of prostaglandin D or its receptors (DPI and CRTH2). Such combinations can be used to treat asthma and other respiratory disorders. Additional examples of therapeutic agents that can be co-administered and/or formulated with one or more anti-PDK1 antibodies or fragments thereof include one or more of: TNF antagonists (e.g., a soluble fragment of a TNF receptor, e.g., p55 or p75 human TNF receptor or derivatives thereof, e.g., 75 kD TNFR-IgG (75 kD TNF receptor-IgG fusion protein, ENBREL)); TNF enzyme antagonists, e.g., TNF converting enzyme (TACE) inhibitors; muscarinic receptor antagonists; TGF-beta antagonists; interferon gamma; perfenidone; chemotherapeutic agents, e.g., methotrexate, leflunomide, or a sirolimus (raparnycin) or an analog thereof, e.g., CCI-779; COX2 and cPLA2 inhibitors; NSAIDs; immunomodulators; p38 inhibitors, TPL-2, MK-2 and NFkB inhibitors, among others.

Notably, the CARs described herein comprising extracellular binding regions including binding domains from anti-PDK1 antibodies, or antigen binding fragments thereof (e.g., scFv) may be used to treat the aforementioned diseases, and may, in certain embodiments, also be used with the combination therapies described herein.

### VII. Pharmaceutical Compositions

The invention also provides pharmaceutical compositions comprising an extracellular PDK1-binding molecule, e.g., antibody, or antigen-binding fragment thereof, of the invention and a pharmaceutically acceptable carrier. The pharmaceutical compositions comprising extracellular PDK1-binding molecules, e.g., antibodies of the invention are for use in, but not limited to, diagnosing, detecting, or monitoring a disorder, in preventing, treating, managing, or ameliorating of a disorder or one or more symptoms thereof, and/or in research. In a specific embodiment, a composition comprises one or more extracellular PDK1-binding molecules, e.g., antibodies of the invention. In another embodiment, the pharmaceutical composition comprises one or more extracellular PDK1-binding molecules, e.g., antibodies of the invention and one or more prophylactic or therapeutic agents other than antibodies of the invention for treating a disorder in which PDK1 (e.g. extracellular PDK1) activity is detrimental , such as cancer, e.g., cancer characterized by expression of extracellular PDK1. Preferably, the prophylactic or therapeutic agents are known to be useful for or having been or currently being used in the prevention, treatment, management, or amelioration of a disorder or one or more symptoms thereof. In accordance with these embodiments, the composition may further comprise of a carrier, diluent or excipient.

The extracellular PDK1-binding molecules, e.g., anti-PDK1 antibodies and antibody-portions of the invention, can be incorporated into pharmaceutical compositions suitable for administration to a subject. Typically, the pharmaceutical composition comprises an extracellular PDK1-binding molecule, e.g., antibody or antibody fragment of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody fragment.

Various delivery systems are known and can be used to administer one or more binding molecules or the combination of one or more binding molecules, e.g., antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing an antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In one embodiment, an antibody of the invention, combination therapy, or a composition of the invention is administered using Alkermes AIR pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., TISSUEL), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent of the invention can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5, 916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gel caps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate) ; or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In a specific embodiment, an antibody of the invention, combination therapy, and/or composition of the invention is administered using Alkermes AIR® pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody fragment) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The extracellular PDK1-binding molecules, e.g., antibodies and antibody-portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an extracellular PDK1-binding molecule, e.g., antibody or antibody fragment of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In other embodiments, an extracellular PDK1-binding molecule, e.g., antibody or antibody fragment of the invention may be conjugated to a polymer-based species such that said polymer-based species may confer a sufficient size upon said binding molecule such that said binding molecule of the invention benefits from the enhanced permeability and retention effect (EPR effect) (See also PCT Publication No. WO2006/042146A2 and U.S. Publication Nos. 2004/0028687A1, 2009/0285757A1, and 2011/0217363A1, and U.S. Pat. No. 7,695,719 (each of which is incorporated by reference herein in its entirety and for all purposes).

Supplementary active compounds can also be incorporated into the compositions. In certain embodiments, an extracellular PDK1-binding molecule, e.g., antibody or antibody fragment of the invention is formulated with and/or co-administered with one or more additional therapeutic agents that are useful for treating disorders in which PDK1 activity is detrimental. For example, an extracellular PDK1-binding molecule, e.g., anti-hPDK1 antibody or antibody fragment of the invention may be formulated and/or co-administered with one or more additional antibodies that bind other targets (e.g., antibodies that bind cytokines or that bind cell surface molecules). Furthermore, one or more binding molecules of the invention may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an extracellular PDK1-binding molecule, e.g., antibody to PDK1 or fragment thereof is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044, which are hereby incorporated by reference for any purpose.

In a specific embodiment, nucleic acid sequences comprising nucleotide sequences encoding an antibody of the invention or another prophylactic or therapeutic agent of the invention are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the invention, the nucleic acids produce their encoded antibody or prophylactic or therapeutic agent of the invention that mediates a prophylactic or therapeutic effect.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990). Detailed description of various methods of gene therapy is provided in US20050042664 A1 which is incorporated herein by reference.

### VIII. Diagnostic Uses of PDK1 Binding Molecules

In another aspect, this application provides a method for detecting the presence of a PDK1-associated disease or disorder, such as a cancer (e.g., a cancer characterized by expression of extracellular PDK1), in a sample in vitro (e.g., a biological sample, such as a tumor tissue sample, or other tissue biopsy, or a cell sample). The subject method can be used to diagnose a disorder, e.g., a cancer. In one embodiment, the method includes: (i) contacting the sample and/or a control sample with an extracellular PDK1-binding molecule, e.g., anti-PDK1 antibody or fragment thereof as described herein; and (ii) detecting formation of a complex between the binding molecule and the sample or the control sample, wherein identification of the formation of the complex in the sample relative to the control sample is indicative of the presence of PDK1 in the sample, e.g., extracellular PDK1 or an extracellular portion of PDK1, and the presence of a PDK1-associated disease or disorder.

In yet another aspect, this application provides a method for detecting the presence of a PDK1-associated disease or disorder, e.g., a cancer, in a subject in vivo (e.g., in vivo imaging in a subject). In one embodiment, the method includes: (i) administering an extracellular PDK1-binding molecule, e.g., anti-PDK1 antibody or fragment thereof as described herein to a subject or a control subject under conditions that allow binding of the binding molecule to PDK1; and (ii) detecting formation of a complex between the binding molecule and PDK1, wherein detection of PDK1 in the subject, e.g.,extracellular PDK1 or an extracellular portion of PDK1, relative to the control subject is indicative of the presence of PDK1 and a PDK1-associated disease or disorder in the subject.

Malignancy of a tumor can be determined using the extracellular PDK1-binding molecules, e.g., antibodies, or antigen-binding fragments thereof, of the invention. In one embodiment, a sample of a tumor obtained from the subject can be tested for the presence of extracellular PDK1 by contacting the sample with an extracellular PDK1-binding molecule, e.g., antibody, or antigen binding fragment thereof, of the invention. The presence of extracellular PDK1 as detected by the binding molecule in the sample indicates that the tumor is malignant. In another aspect, the invention provides a method for determining whether a cancer in a subject is metastatic, comprising determining the presence of extracellular PDK1 in a sample from the subject by contacting the sample with an extracellular PDK1-binding molecule, e.g., antibody, or antigen binding fragment thereof, of the invention and detecting the presence of extracellular PDK1 in the sample. The presence of extracellular PDK1 as detected by the binding molecule in the sample indicates that the cancer is metastatic.

The invention also provides a method of identifying a subject having cancer which is responsive to anti-PDK1 treatment, comprising determining the presence of extracellular PDK1 in a sample from the subject by contacting the sample with the extracellular PDK1-binding molecule, e.g., antibody, or antigen binding fragment thereof, of the invention, and detecting the presence of extracellular PDK1 in the sample. The presence of extracellular PDK1 detected by the binding molecule in the sample indicates that cancer will be responsive to an anti-PDK1 treatment. In one embodiment, the anti-PDK1 treatment comprises administering an extracellular PDK1-binding molecule, e.g., antibody, or antigen binding fragment thereof, of the invention.

The invention also provides a method of identifying a subject having cancer which is responsive to an extracellular PDK1-binding molecule, e.g., antibody, or antigen binding fragment thereof, of the invention, said method comprising determining the presence of extracellular PDK1 in a sample from the subject, wherein the presence of extracellular PDK1 in the sample indicates that cancer will be responsive to treatment with the binding molecule of the invention.

In various embodiments, the cancer may be breast cancer, cervical cancer or squamous cell carcinoma. In other embodiments, the cancer is metastatic or non-resectable.

Given their ability to bind to PDK1, the extracellular PDK1-binding molecules, e.g., anti-PDK1 antibodies, or antigen-binding fragments thereof, of the invention can be used to detect PDK1, e.g., extracellular PDK1 or extracellular PDK1, for example in a biological sample, such as tumor tissue, cells, serum or plasma, using a conventional immunoassay, such as an enzyme linked immunosorbent assays (ELISA), an radioimmunoassay (RIA) or tissue immunohistochemistry.

In one embodiment, the extracellular PDK1-binding molecule, e.g., antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

The binding molecule can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting. Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter. Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example: (i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (ii) alkaline phosphatase (AP) with para-Nitrophenyl phosphate as chromogenic substrate; and (iii) .beta.-D-galactosidase (.beta.-D-Gal) with a chromogenic substrate (e.g., p-nitrophenyl-.beta.-D-galactosidase) or fluorogenic substrate 4-methylumbelliferyl-.beta.-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the binding molecule. The skilled artisan will be aware of various techniques for achieving this. For example, the binding molecule can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the binding molecule, the binding molecule is conjugated with a small hapten (e.g., digoxin) and one of the different types of labels mentioned above is conjugated with an anti-hapten antibody (e.g., anti-digoxin antibody). Thus, indirect conjugation of the label with the binding molecule can be achieved.

In another embodiment of the invention, the binding molecule need not be labeled, and the presence thereof can be detected using a labeled antibody which binds to the binding molecule.

Alternative to labeling the binding molecule, human PDK1 can be assayed by a competition immunoassay utilizing rhPDK1 standards labeled with a detectable substance and an unlabeled anti-human PDK1 antibody. In this assay, the biological sample, the labeled rhPDK1 standards and the anti-human PDK1 antibody are combined and the amount of labeled rhPDK1 standard bound to the unlabeled antibody is determined. The amount of human PDK1 in the biological sample is inversely proportional to the amount of labeled rhPDK1 standard bound to the anti-PDK1 antibody. Similarly, human PDK1 can also be assayed by a competition immunoassay utilizing rhPDK1 standards labeled with a detectable substance and an unlabeled anti-human PDK1 antibody.

The binding molecules, e.g., antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyze is bound by a first antibody which is immobilized on a solid support, and thereafter a second antibody binds to the analyze, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tumor sample may be fresh or frozen or may be embedded in paraffin and fixed with a preservative such as formalin, for example.

The binding molecules, e.g., antibodies may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P or ³⁵S), or a dye so that the tumor can be localized using immunoscintiography.

As a matter of convenience, the binding molecule of the present invention can be provided in a kit, i.e., a packaged combination of reagents in predetermined amounts with instructions for performing the diagnostic assay. Where the binding molecule is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### IX. Kits

The invention also provides compositions and kits for diagnosing a malignancy or cancer, recurrence of a cancer, or survival of a subject being treated for cancer e.g. a cancer characterized by expression of extracellular PDK1. These kits include one or more of the following: a detectable antibody that specifically binds to extracellular PDK1 (e.g., 2B9) and one or more of a detectable antibody that specifically binds to the extracellular PDK1 antibody, reagents for obtaining and/or preparing subject tissue samples for staining, and instructions for use.

The invention also encompasses kits for detecting the presence of extracellular PDK1 in a biological sample. Such kits can be used to determine if a subject is suffering from or is at increased risk of developing cancer, e.g., a metastatic or non-resectable cancer, and/or a cancer that is characterized by expression of extracellular PDK1. For example, the kit can comprise a labeled compound or agent capable of detecting extracellular PDK1 in a biological sample (e.g., an antibody) and optionally means for determining the amount of the extracellular PDK1 in the sample (e.g., an antibody). Kits can also include instructions for use of the kit for practicing any of the methods provided herein or interpreting the results obtained using the kit based on the teachings provided herein. The kits can also include reagents for detection of a control protein in the sample not related to the cancerous state, e.g., actin for tissue samples, albumin in blood or blood derived samples for normalization of the presence or amount of the marker present in the sample. The kit can also include the extracellular PDK1 for detection for use as a control or for quantitation of the assay performed with the kit.

For antibody-based kits, the kit can comprise, for example: (1) a first antibody (e.g., attached to a solid support) which binds to a first marker protein, e.g., extracellular PDK1; and, optionally, (2) a second, different antibody which binds to either the first marker protein or the first antibody and is conjugated to a detectable label.

Reagents specific for detection of a marker of the invention, e.g., extracellular PDK1, allow for detection and/or quantitation of the marker in a complex mixture, e.g., serum, tissue sample. In certain embodiments, the reagents are species specific. In certain embodiments, the reagents are not species specific. In certain embodiments, the reagents are isoform specific. In certain embodiments, the reagents are not isoform specific.

The kits of the invention may optionally comprise additional components useful for performing the methods of the invention. By way of example, the kits may comprise fluids (e.g., SSC buffer, TBST) suitable for annealing complementary nucleic acids or for binding an antibody with a protein with which it specifically binds, one or more sample compartments, an instructional material which describes performance of a method of the invention and tissue specific controls/standards. In certain embodiments, the kits can also comprise, e.g., a buffering agents, a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate. Each component of the kit can be enclosed within an individual container and all of the various containers can be within a single package, along with instructions for interpreting the results of the assays performed using the kit.

The kits of the invention may optionally comprise additional components useful for performing the methods of the invention.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLES

### Example 1

### Isolation of the Human mAb 2B9 from Breast Cancer Lymph Node B-cells and Binding of 2B9 to PDK1

An anti-PDK1 human antibody (referred to herein as “2B9”) was identified using methods described in Dessain et al., 2004, J Immunol Methods 291(1-2): p. 109-22; Adekar et al., 2009, J Immunol Methods 333(1-2): 156-166; and U.S. Pat. Nos. 7,491,530 and 8,557,575, and as described below. Hybrid cells that secrete human monoclonal antibodies (mAbs) that originated within the B cells isolated from the lymph nodes were produced using the methods described in U.S. Pat. Nos. 7,491,530 and 8,557,575. This is accomplished through the use of the B5-6T fusion partner cell line, which expresses an ectopic human telomerase gene that stabilizes human chromosomes in the hybrid cells. Hybrid cells made from the B5-6T cell line and primary human B-cells stably express human mAbs. Thus, this method uses a telomerase-stabilized fusion partner cell line to produce hybridomas secreting IgGs without the need for recombinant mAb production.

Briefly, mononuclear cells from tumor-draining lymph nodes from breast cancer patients were obtained from the University of Vermont Cancer Center. Patient lymph nodes were shipped overnight in PBS at 4° C. Mononuclear cells were isolated from whole lymph nodes, followed by isolation of CD27+ cells with anti-CD27 magnetic beads (Miltenyi Biotec, Auburn, Calif.) according to manufacturer's instructions. CD27+ cells were cultured for 8 days in RPMI-1640 supplemented with 10% Benchmark FBS (Gemini Bio-Products, Sacramento, Calif.), Human UltraCD40L (Multimeric BioTherapeutics, San Diego, Calif.), and additional growth factors, at 37° C. Day 8 cultured cells were electrofused with the B5-6T heteromyeloma cell line and selected with HAT (Sigma-Aldrich) in Advanced RPMI-1640+1% FBS.

Post-selection hybridoma supernatants were screened by a whole cell immunofluorescence binding assay. MCF7, T47D, MDA-MB-231, and A431 cancer cell lines were seeded into 96-well plates at 5,000 cells/well in their respective growth medium and cultured for 3 days in a 37° C. humidified incubator at 5% CO₂. Cells were washed with PBS and fixed with 2% methanol-free formaldehyde (Thermo Scientific, Rockford, Ill.) for 30 minutes and washed twice with PBS. Hybridoma supernatants were incubated on the cells for 1 hour in a 5% CO₂ incubator at 25° C. then cells were washed twice with PBS. Secondary antibody (Alexa Fluor® 488-conjugated Goat Anti-Human IgG [H+L]; Jackson ImmunoResearch, West Grove, Pa.) diluted 1:1,000 and Hoechst nuclear stain (Pierce Biotechnology) 1:1,000 in PBS was incubated on the cells for 1 hour then cells were washed twice with PBS. Supernatants containing cell-reactive Human IgG were detected and imaged by the Operetta (University of Pennsylvania Wistar Institute).

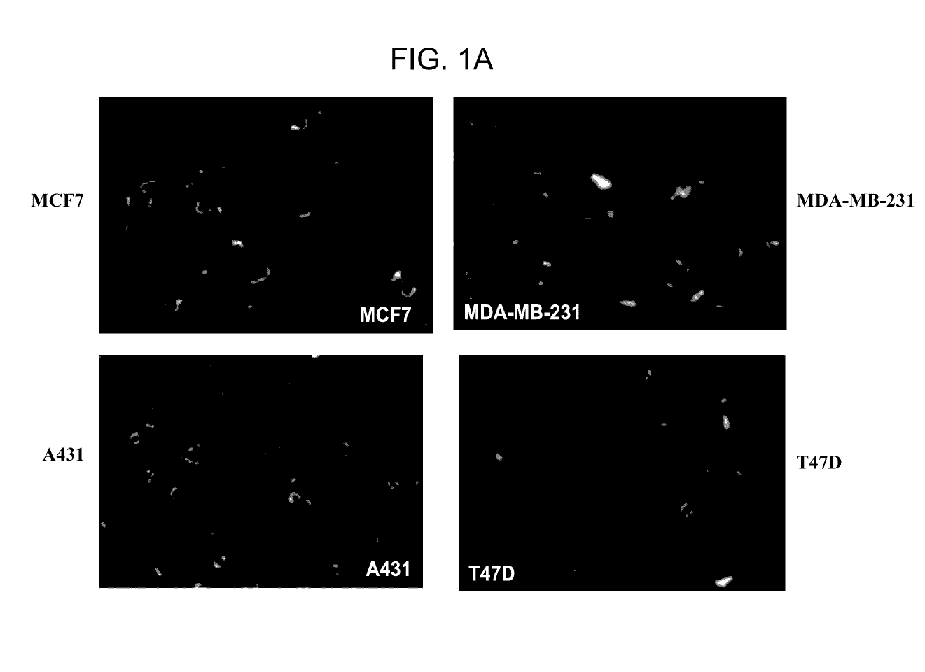

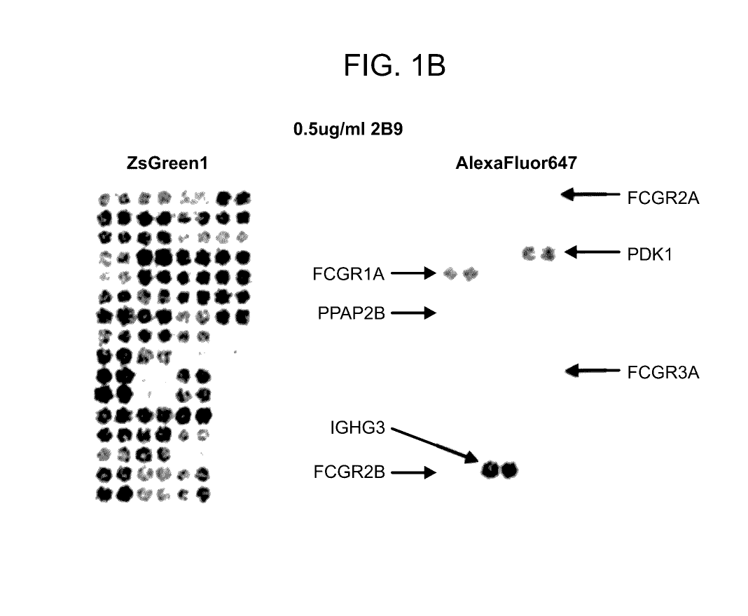

Cell-reactive Human IgG secreting hybridomas were stabilized for cell reactivity/binding by limiting dilution cloning in liquid medium followed by detection in the whole cell immunofluorescence assay using a Goat Anti-Human IgG, Fc Specific (Invitrogen) secondary antibody and detected and imaged by Operetta for two consecutive rounds. Stabilized cell-reactive Human IgG secreting hybridomas were then cloned by limiting dilution in semi-solid medium containing 1:1,000 dilution of Human IgG (H+L) specific fluoroscein (CloneDetect) to isolate and stabilize hybridoma colonies secreting the highest amount of cell-reactive Human IgGs identified by the ClonePix. Stabilized, cell-reactive high Human IgG secreting hybridomas were expanded into 24-well plates and concentrated supernatants were screened for whole cell live Immunofluorescence binding and live cell FACS binding to MCF7, T47D, MDA-MB-231, and A431 cancer cells using Goat Anti-Human IgG (H+L) and Goat Anti-Human Fc-Specific secondary antibodies. The 2B9 mAb bound to each of the cancer cell lines as described in FIG. 1A, including three different breast cancer cell lines and a cervix squamous cell carcinoma cell line, The 2B9 antibody did not bind to non-malignant HEK293 cells (data not shown). In addition, the 2B9 antibody bound to the extracellular membrane of A549 cells (lung cancer, and the breast cancer cell lines SK-BR-3, MDA-MB-231, and MCF-7 cell lines, as determined by immunofluorescence (FIG. 1A).

Hybridomas secreting IgGs which bound to one or more live cancer cell lines by either method were adapted to Advanced RPMI-1640 medium with 5% Ultra Low IgG fetal bovine serum (Life Technologies, Grand Island, NY) and expanded up to a 2L roller bottle. Filtered supernatants were purified over a protein L column. Antibody concentrations were determined using the NanoDrop spectrophotometer (NanoDrop Technologies, Wilmington, DE).

Isotyping was performed by ELISA. ELISA plates were coated with rabbit anti-human IgG (H&L) (Southern Biotechnology, Birmingham, AL). The plates were used to bind IgG from hybridoma-conditioned media. The captured IgGs were reacted with mouse anti-human isotype and light chain specific antisera conjugated to horseradish peroxidase (Sigma-Aldrich, St. Louis, Mo.). mAb 2B9 was found to be IgG1-K.

To determine the 2B9 antigen, 2B9 was analyzed using the Retrogenix gene expression array, in which HEK293 cells are overlaid on a glass slide spotted with plasmids expressing membrane-associated proteins. For primary screening, 3559 expression vectors, each encoding a full-length human plasma membrane protein, were arrayed in duplicate across 10 microarray slides. Of the 3559 human plasma membrane proteins screened, 2625 are unique genes. An expression vector (pIRES-hEGFR-IRES-ZsGreenl) was spotted in quadruplicate on every slide, and was used to ensure that a minimal threshold of transfection efficiency had been achieved or exceeded on every slide. This minimal threshold (mean ZsGreen signal from the pIRES-EGFR-ZsGreen vector over background of 1.5) has been defined previously. Human HEK293 cells were used for reverse transfection/expression. The 2B9 mAB was bound at to the cell monolayer and detection of binding was performed by using the same fluorescent secondary antibody as used in the background screen. Fluorescent images were analysed and quantitated (for transfection efficiency) using ImageQuant software (GE). A protein ‘hit’ was defined as a duplicate spot showing a raised signal compared to background levels. This is achieved by visual inspection using the images gridded on the ImageQuant software. Using this method, the target for 2B9 was determined to be PDK1 (FIG. 1B).

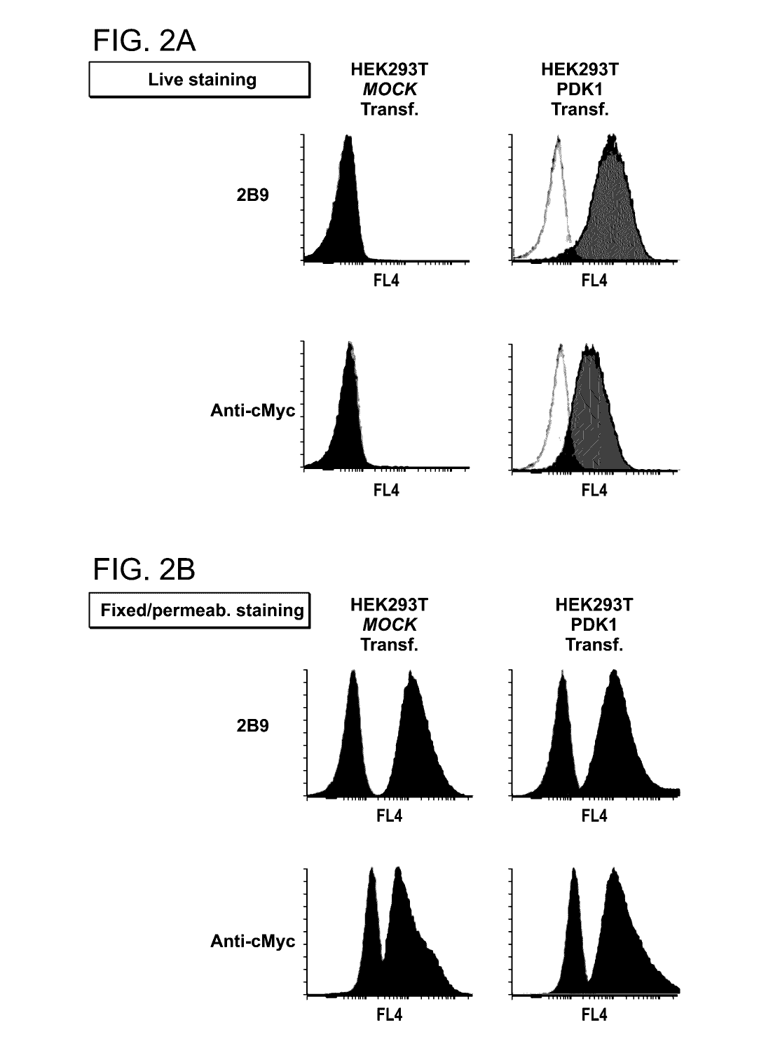

Confirmation of the PDK1 antigen on the outer plasma membrane of cells for antibody 2B9 was performed using 293T cells that were transfected with an N-terminally myc-tagged recombinant PDK1 gene and assessed by flow cytometry. In the transfected cells, the recombinant PDK1 protein was detectable on the outer plasma membrane, as shown by binding of 2B9 and anti-myc antibodies on the surface of live (unpermeabilized) transfected cells, but not on live, mock-transfected cells (FIG. 2A). Both the 2B9 and the anti-myc antibodies bound antigen in permeabilized cells, whether transfected with the recombinant 2B9 gene construct or mock-transfected (FIG. 2B).

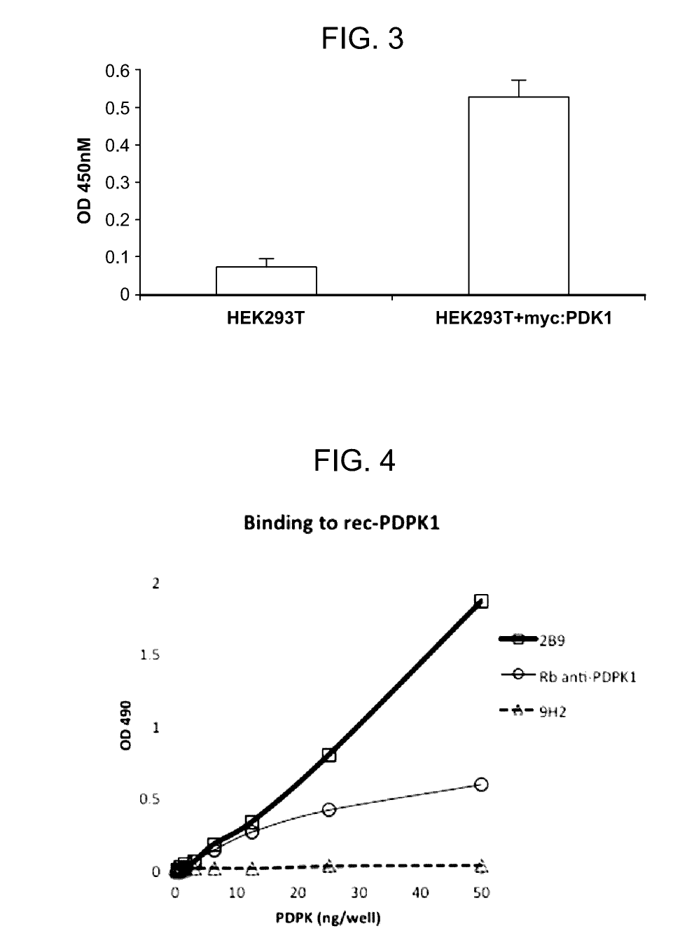

Antigen specificity for antibody 2B9 was also assessed using a standard sandwich ELISA assay. The results are shown in FIGS. 3 and 4 and confirm that PDK1 is the antigen bound by antibody 2B9.

In the experiment in FIG. 3, HEK293T cells were transfected with myc tagged PDK1 construct (myc:PDK1). After 48 hours, approximately 1 million of myc:PDK1 transfected HEK293T cells or untransfected HEK293T control cells were collected, washed with PBS, then lysed with 1 ml of radioimmunoprecipitation assay buffer (RIPA buffer), respectively. 50 ul of the cell lysates were added into ELISA plate wells coated with anti-myc tag antibody 9E10 (Santa Cruz Biotech #sc-40) and incubated for 1 hour in room temperature. mAb 2B9 (1 ug/ml) was used as primary antibody for detection. Goat anti-human IgG HRP (Southern Biotech #9040, 1:5000) was used as secondary antibody for detection.

Further confirmation is provided in the data shown in FIG. 4, which describes the binding of the 2B9 antibody to recombinant PDK1 protein. Serial dilutions of recombinant PDPK1 protein (SignalChem's active PDK1 #P14-10H, baculovirus sf9) were used to coat ELISA plate wells. Bound PDPK1 was tested for binding of purified mAb 2B9. Controls included the commercial rabbit anti-PDPK1 (Cell Signaling #3062P, raised against a C-terminal peptide), and the IgG control monoclonal antibody 9H2 (does not bind PDK1). Goat anti-human IgG-HRP (Southern Biotech #9040, 1:5,000) was used as secondary antibody for human mAbs 2B9 and 9H2 and donkey anti-rabbit IgG-HRP (Jackson Immunoresearch, at 1:5,000) was used for the commercial rabbit antibody. The commercial anti-PDK1 and the 2B9 mAb bound to the plate-bound PDK1, but the control mAb did not, indicating that the 2B9 mAb binds PDK1.

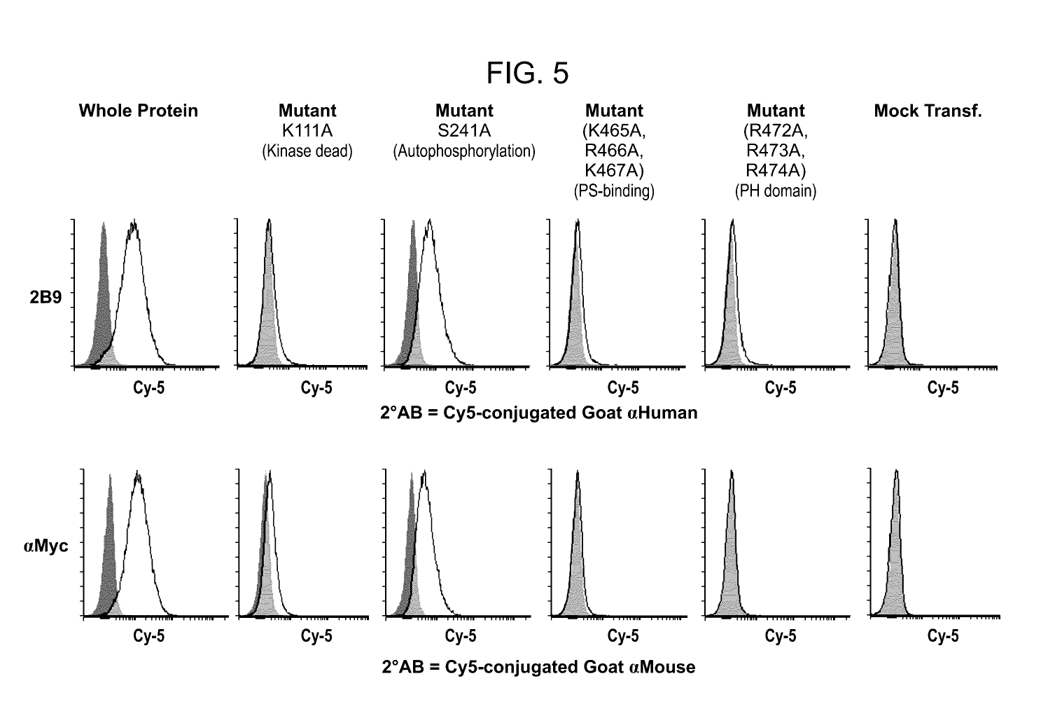

Cell surface expression of PDK1 on transfected 293T cells was detected using flow cytometry. DNAs containing different PDK1 cDNAs were transfected into 293T cells and surface expression of 2B9 antigen (PDK1) was analyzed by flow cytometry on live (unpermeabilized) cells. The constructs also encoded an N-terminal myc tag, enabling dectection with the 9E10 mAb. MAb binding was detected with a Cy5-conjugated goat secondary antibody (anti-human for 2B9, anti-mouse for 9E10). Transfected genes encoded wild-type, kinase dead (K111A), autophosphorylation site dead (S241A), phosphatidyl serine pocket mutated (K465A, R466A, K467A) and pleckstrin-homology domain mutated (R472A, R473A, R474A) forms. Expression of the mutant proteins was confirmed in permeabilized cells (data not shown). Notably, the kinase dead mutant (K111A) was not displayed on the surface, even though it was expressed intracellularly (data not shown). This suggests that PDK1 kinase activity initiates downstream events that lead to PDK1 relocation to the cell surface, potentially through phosphorylation of Akt. The lack of surface expression of the mutant proteins (see FIG. 5) suggests that PDK1 phosphorylation and interaction with cell membrane lipids are important features of extracellular PDK1 expression.

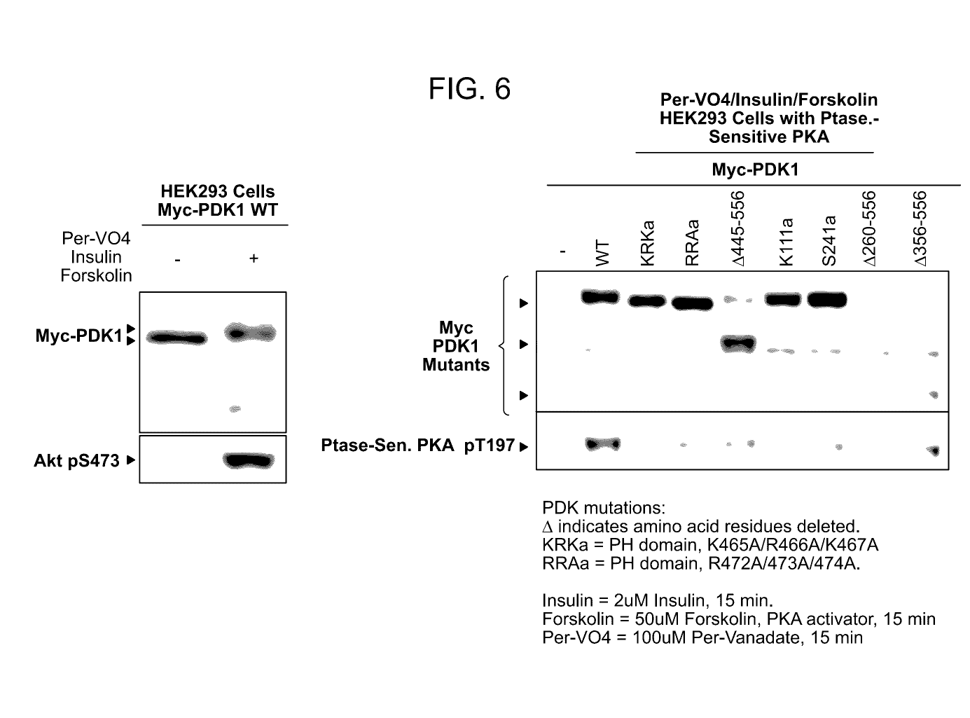

Mutant versions of PDK1 were studied to determine whether such mutant proteins could be expressed by transfected cells. Specifically, four different PSK1 mutant forms were expressed in HEK293 cells, including the KRK-A PDK1 mutant which has three mutations in the PH domain (K465A/R466A/K467A); the RRA-A PDK1 mutant which has three mutation in the PH domain (R472A/R473A/R474A); and the S241A PDK1 mutant, which alters the autophosphorylation site on PDK1. Deletion variants of PDK1 were also expressed, including the Δ445-556 PDK1 variant; the Δ260-556 PDK1 variant, and the Δ356-556 PDK1 variant. All of the mutated proteins fail to localize on the outer plasma membrane, except for the S241A mutant, even though the mutant proteins were highly expressed in the transfected cells, as shown in the Western blot in FIG. 6. This indicates that specific functional domains are required for localization of PDK1, when overexpressed by transfection, on the outer plasma membrane, suggesting that an intact kinase domain and lipid interaction domains play a role in their transport to and/or maintenance at that location.

Plasma membrane expression of PDK1 was quantified in cells transfected with myc-labeled PDK1, as well as variants thereof, and analyzed using the In Cell Western method with the LI-COR imager. HEK293 cells were transfected with Myc-PDPK1 wild type (WT) and Myc-PDPK1 PH domain mutants (PH mutants). The PDK1 PH domain mutants that were tested included Mutant 1: myc-PDPK1, K465A-R466A-K467A (KRK-A mutant) mixed with Mutant 2: myc-PDPK1, R472A-R473A-R474A (RRR-A mutant). Cells in wells were fixed with and without permeabilization, where culturing conditions included a plate of 50,000 cells on 48-well plate, and the expressed proteins were detected with the flourescent anti-myc antibody. Although the amounts of recombinant protein expressed in the cells were approximately equal, as detected in the permeabilized cells, the amount of PH domain mutant proteins localized on the outer membrane of intact cells was reduced by approximately half, compared to the wild-type PDK1 proteins. This suggests that the localization of the PDK1 on the outer plasma membrane is mediated by interactions between the PH domain and membrane lipids. Quantitation is described in Table 2 below:

TABLE 2
Quantitation of plasma membrane expression of cells
transfected with Myc tagged PDK1

| | Wild Type | PH Mutants: KRK-A, |
|---|---|---|
| | (WT) | and RRR-A |
| Anti-Myc Non-Permeabilized | 48 | 21 |
| Anti-Myc Permeabilized | 404 | 435 |

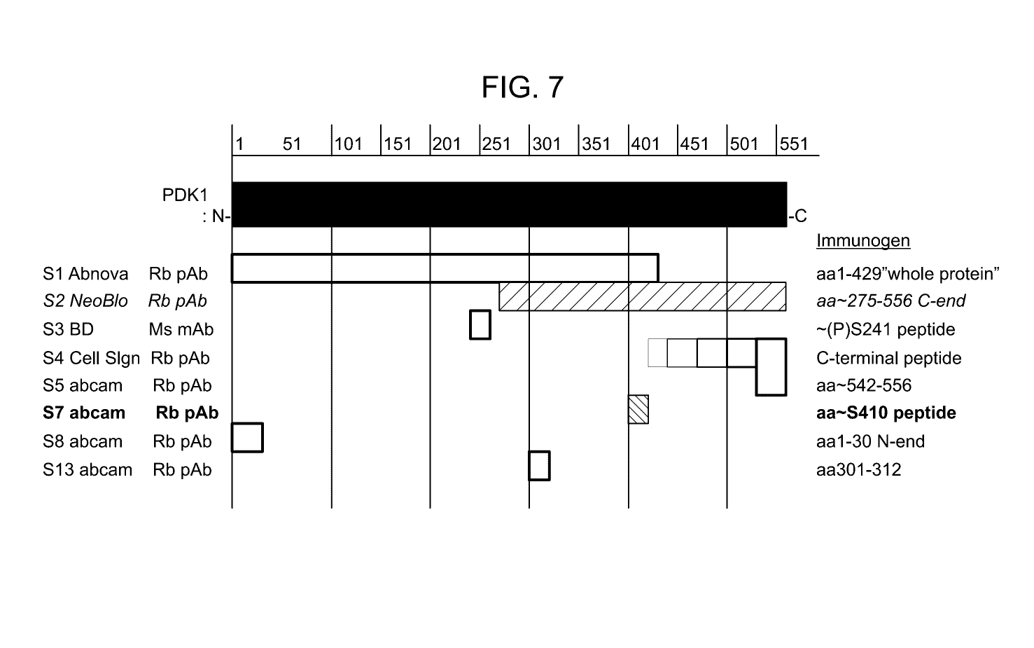

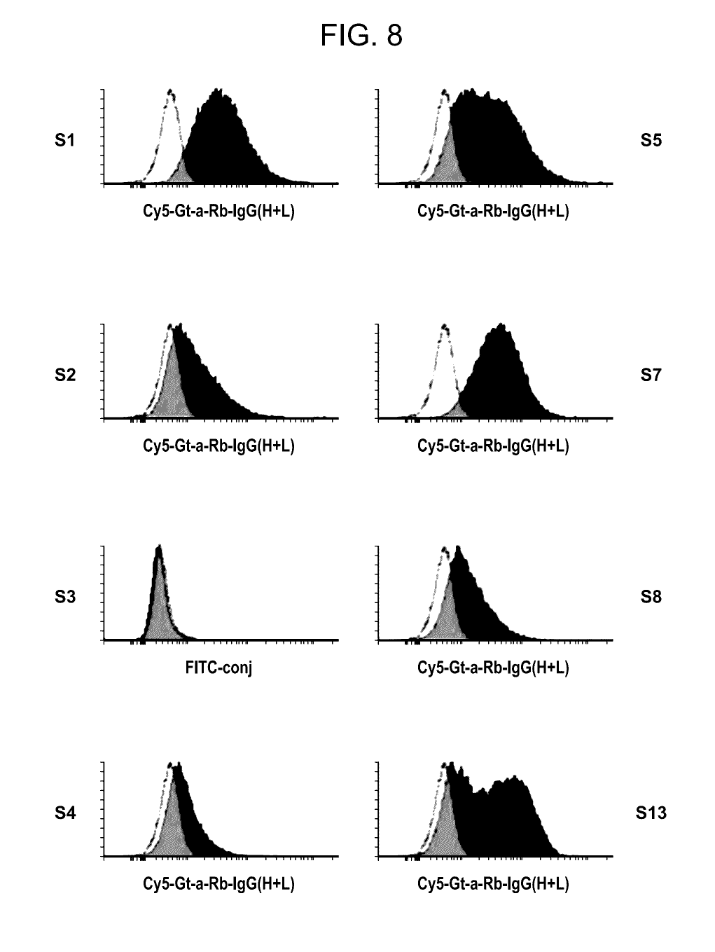

FIGS. 7 and 8 show the results of experiments in which commercially available anti-PDK1 antibodies were tested for binding to outer plasma membrane associated PDK1. The antibodies were tested for binding to intact K562 cells by flow cytometry. FIG. 7 is a summary of the antibodies used, combined with a depiction of the PDK1 antigens (Immunogen) used to create the antibodies. The commercial sources are shown at the left, the immunogens are listed at the right, and the locations of the immunogens, relative to the sequence of the PDK1 protein, are depicted in the horizontal bars shown below the PDK1 sequence above. The antibodies are also assigned a code (S1-S5, S7, S8, S13), which is used to identify them in FIG. 8.

FIG. 8 provides results using flow cytometry to assess the ability of the commercial antibodies shown in FIG. 7 to bind to intact K562 cells (human myelogenous leukemia cells). All of the commercially available antibodies bound to the cell line except S3, an Abcam monoclonal antibody specific for phosphorylated S241 residue on the protein. This corresponds to the amino acid that, when mutated to an alanine, had no effect on extracellular localization. Taken together, these results suggest that 5241 phosphorylation is not required for extracellular expression. The binding of the other commercial antibodies to the human leukemia cell line supports the observation that PDK1 is expressed on the outer plasma membrane in some cells. The observation that both N-terminal and C-terminal-specific antibodies (S8 and S5, respectively) bound extracellular PDK1 suggests that the entire protein is on the outer plasma membrane and therefore available for binding by extracellular antibodies or polypeptides.

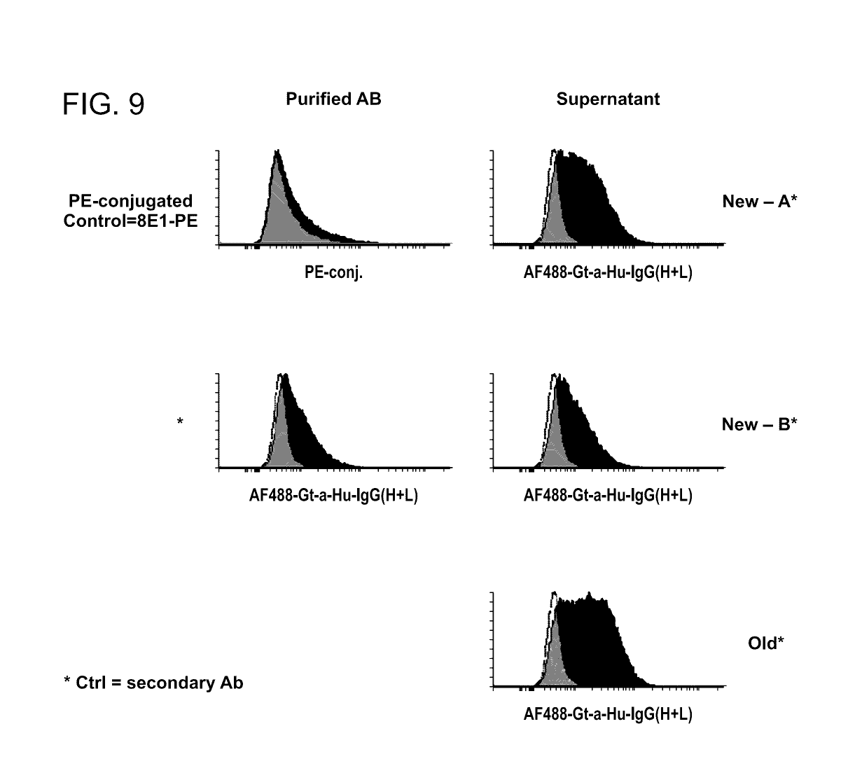

FIG. 9 shows the results of experiments to assess binding of a number of 2B9 preparations on K562 cells. Binding to K562 cell lines was assessed by flow cytometry and was compared to binding of the antibody 8E1, an isotype control human mAb. Two mAb preparations (left panels) and three 2B9 hybridoma cell culture supernatants were tested for binding by flow cytometry. In each case, signal is greater than that seen with the isotype control antibody.

### Example 2

### Assessing the Potential Anti-Metastatic Function of 2B9 In Vitro by Cell Invasion Assay

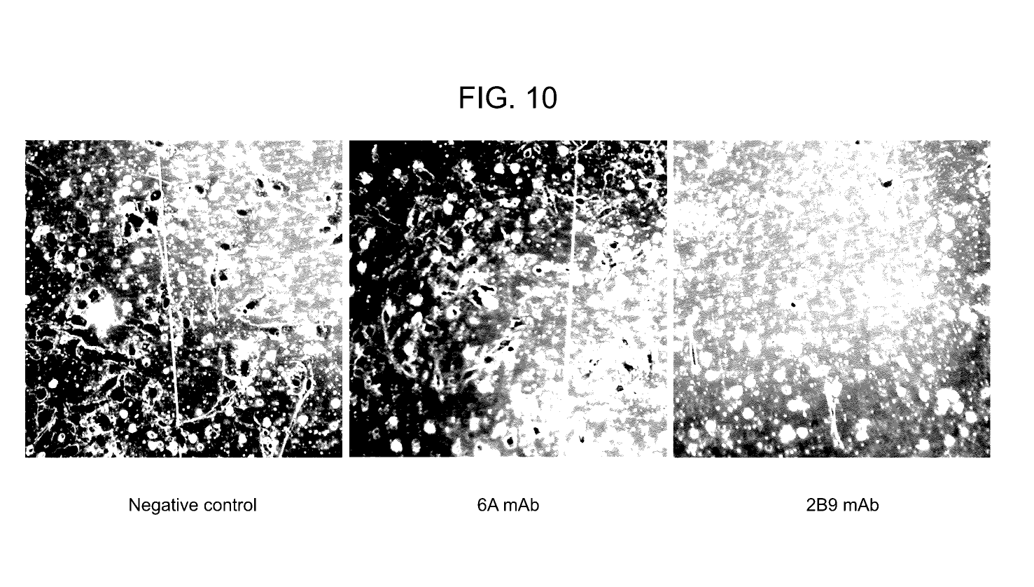

In vitro cell migration and invasion assays were used to determine if 2B9 interferes with the motility of PDK1 expressing MDA-MB-231 cells (a human breast epithelial adenocarcinoma cell line that is metastatic). Briefly, MDA-MB-231 cells were grown to 80% confluence and then were serum starved in the presence or absence of 2B9 or an isotype control antibody 6A. 24 hours later, 50,000 MDA-MB-231 cells from each group were re-suspended in serum free L-15 medium+/−10 ug/ml 6A or 2B9 then seeded onto the upper chamber of 24-well Matrigel Invasion chambers (Corning) (n=3). The chambers were incubated at 37° C., atmospheric CO₂. 24 hours later, non-invading cells in the upper portion of the chambers were wiped away with cotton swabs. The invading cells were fixed with 4% paraformaldehyde for 30 minutes and rinsed with DPBS. Fixed cells were then stained with crystal violet dye solution for 20 minutes and residual dye was rinsed away with washes in a beaker of dH₂0. Chambers were allowed to air dry for 30 minutes then images of invasion chambers Negative Control (A), Antibody 6A (B), and Antibody 2B9 (C) were captured at 100× magnification (FIG. 10). As shown in FIG. 10, MDA-MB-231 cells treated with mAb 2B9 exhibited reduced cell invasion relative to the controls. The results indicate the utility of 2B9 for inhibiting cancer cell migration.

### Example 3

### Identification of Potential Mechanisms for Anti-Tumor Activity of mAb 2B9

Potential mechanisms of 2B9 mAb killing of PDK1-positive cell lines are tested, including antibody-dependent cellular cytotoxicity (ADCC) and complement-dependent cytotoxicity (CDC). Potential killing mechanisms are assessed using 2B9 mAb and cells with high levels of PDK1 antigen expression, such as MDA-MB-231, as well as 293T cells that stably express PDK1 (FIG. 1) from a transduced lentiviral vector positively selected for blasticidin expression (DNASU Plasmid Repository, Tempe, Ariz.).

To test CDC, such as is operative for anti-CD20 mAbs or combinations of anti-EGF receptor mAbs, a non-radioactive complement lysis assay is used (Dechant, et al., 2008, Cancer Res, 2008. 68(13): p. 4998-5003; Gazzano-Santoro, et al., 1997, J Immunol Methods 202(2): 163-71). Cells are incubated with a titration series of mAb 2B9 (0 & 10-1000 μg/ml) and 1/5 diluted rabbit complement at 37° C. for 1 hour. Cell viability is assessed using Almar blue dye or the LIMR HEDS assay (Li, et al., 2012, Toxicol In Vitro 26(4): p. 603-12). A CDC assay from Cell Technologies may also be used.

To evaluate ADCC activity, such as is operative for anti-Her2/neu receptor mAbs, the aCella-TOX assay (Cell Technology, Fremont, Calif.) is used according to manufacturer's instructions. Target cell lysis is detected by measuring release of endogenous glyceraldehyde-3-phosphate dehydrogenase. NK cells are collected from peripheral blood mononuclear cells and cryopreserved using the NK cell isolation kit (Miltenyi Biotec, Cologne). The optimized effector:target cell ratio is determined in control experiments, (i.e. NK cells: MDA-MB-231 cells). Briefly, serial dilutions of 2B9 mAb (0 & 10-1000 μg/ml) are added in a 25 μL volume to target cells (2×10⁵ cells/mL in 25 μL media in 96-well plates) and allowed to opsonize for 15 minutes at 37° C. NK cells are added at two pre-determined E:T ratios and plates are centrifuged for one minute, and incubated at 37° C. for 1.75 hours. Plates are then incubated at room temperature for 5-10 min. Controls include a maximum lysis control, effector cell only, and target cell only. All conditions are performed in triplicate. Cell lysis is measured by assaying luciferase activity induced by a coupling reagent per manufacturer's instruction. The relative luciferase units are graphed and the data reduced by four-parameter fits for analysis. Total cytotoxicity % is calculated by dividing adjusted sample well signal by the adjusted average maximum lysis signal, and multiplying the result by 100. The DELFIA TRF ADCC assay (PerkinElmer, Waltham, Mass.) may also be used. Standardized human NK cells from STEMCELL Technologies (Vancouver) may also be used for NK cell standardization.

Differences in CDC and ADCC activity are determined by comparing isotype control values obtained to 2B9 values, and a Student's t-test is used for the analysis of differences between groups. A probability (P) value of <0.05 is considered significant. If 2B9 elicits significant toxicity by CDC or ADCC (p <0.05) it is tested as an un-modified mAb in animal models of metastasis as described below in Example 4.

### Example 4

### Testing the Efficacy of 2B9 Anti-PDK1 Antibody Therapeutic in Mouse Models of Metastatic Breast Cancer

The 2B9 mAb is tested for its therapeutic potential in vivo in a human breast cancer xenograft model of metastasis. Both orthotopic and metastatic breast cancer models are tested using the metastatic human breast epithelial adenocarcinoma cell line MDA-MB-231. Mouse models of breast cancer are described, for example, in Wang, et al., 2011, J Pharmacol Exp Ther, 2011. 339(2): 421-9; Zhao, et al., 2010, J Ultrasound Med 29(4): 587-95; and Kuperwasser, et al., 2005, Cancer Res 65(14): 6130-8. For the orthotopic metastatic breast cancer model, female NOD SCID mice are injected orthotopically in mammary fat pads with 1×10⁶ MDA-MB-231 cells harvested at exponential growth phase and suspended in diluted Matrigel 1:3 (BD Biosciences, Bedford, Mass.). Injection volumes are 40 μL using a 31-gauge needle and 50 μL Hamilton syringe (Kuperwasser, et al., 2005, Cancer Res 65(14): 6130-8).

Immunodeficient NOD/SCID and NSG mice age 8-10 weeks are housed under aseptic sterile conditions and provided autoclaved food and water ad libitum. Surgeries are performed under sterile conditions and animals receive antibiotics (Trimethoprim Sulfa) in drinking water for 2 weeks following surgery.

Each experimental group consists of 10 mice and each treatment consists of 0, 100 or 250 pg of 2B9 mAb or isotype match control mAb. Two different dosing strategies are used. In the first strategy, 2B9 mAb is administered, starting on the day of the initial tumor inoculation, every other day. In the second strategy, tumors are allowed to establish a volume of 0.5 cm². At that time, mAb injections commence and continue every other day.

Mice are monitored daily for tumor volume using digital calipers, and twice daily if morbidity ensues (poor body condition, ulcerating tumor, hypokinesis, changes in fur and/or eyes), and are euthanized if tumors reach 10% of body weight. Tumor volume is calculated using the ellipsoid formula. At the time of necropsy, 43 days post injection, tumor size & weight in the orthotopic site is measured and quantification of the percentage of mice bearing macrometastasis to the lung is performed. The metastatic lung nodules over 1 mm in diameter are counted after fixation of excised lungs in 10% (v/v) formalin for 24 hr.

Orthotopic tumor volume, percentage of mice bearing macrometastasis, and number of metastatic lung nodules is calculated for each treatment group. Results are expressed as the mean±SE. Statistical significance of differences between groups is assessed using a Student's t-test and p <0.05 is considered significant and indicates that mAb 2B9 has anti-tumorigenic and/or anti-metastatic function in vivo.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent, patent application, and GenBank number referred to in the instant application is hereby incorporated by reference as if each reference were noted to be incorporated individually.

### SEQUENCE SUMMARY

| | | |
|---|---|---|
| Sequence | Nucleic Acid or | |
| Identifier | Protein | Sequence |

Hybridoma Clone 2B9 (anti-PDK1)

| | | |
|---|---|---|
| SEQ ID NO: 1 | Heavy chain 2B9 | GGATTTGGGCTGCGCTGGGTTTTTCTTGTGGCTATTTTAAAAGGTGTCCAGTGTGAGGTGCAGCT |
| | nucleic acid | GTTGGAGTCAGGGGGAGGCTTGGTACAGCCGGGGGGGTCCCTGAGACTCTCCTGTGCAGCCTCTG |
| | | GGTTCACCTTTAGTAACTTTGGCATGACCTGGGTCCGCCAGGCTCCGGGGAAGGGGCTGGAGTGG |
| | | GTCTCAGGTGTAAGTATTAGGGGCATTGCCACATACTACGCAGACTCCGTGAAGGGCCGGTTCAC |
| | | CATCTCCAGGGACGATTCCAGGAACACGTTGTATCTACAAATGAACAGCCTGAGAGCCGAGGACA |
| | | CGGCCCTTTATTACTGTGCGACCGGTGTTAGACTCCTTGGGCGTGAGTGGGGCCAGGGAACCCTG |
| | | GTCACCGTCTCCTCGGCCTCCACCAAGGGCCCATCGGTCTTCCCCCTGGCACCCTCCTCCAAGAG |
| | | CACCTCTGGGGGCACAGCGGCCCTGGGCTGCCTGGTCAAGGACTACTTCCCCGAACCGGTGACGG |
| | | TGTCGTGGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTTGAGCCCAAATCTTGTG |
| | | ACAAAACTCACACATGCCCACCGTGCCCAGCACCTGAACTCCTG |
| SEQ ID NO: 2 | Heavy chain 2B9 | GFGLRWVFLVAILKGVQCEVQLLESGGGLVQPGGSLRLSCAASGFTFSNFGMTWVRQAPGKGLEW |
| | protein | VSGVSIRGIATYYADSVKGRFTISRDDSRNTLYLQMNSLRAEDTALYYCATGVRLLGREWGQGTLV |
| | | TVSSASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPELL |
| | | [Constant region is italicized] |
| SEQ ID NO: 3 | Heavy chain | GGATTTGGGCTGCGCTGGGTTTTTCTTGTGGCTATTTTAAAAGGTGTCCAGTGTGAGGTGCAGCTGT |
| | variable region- | TGGAGTCAGGGGGAGGCTTGGTACAGCCGGGGGGGTCCCTGAGACTCTCCTGTGCAGCCTCTGGGTT |
| | 2B9 | CACCTTTAGTAACTTTGGCATGACCTGGGTCCGCCAGGCTCCGGGGAAGGGGCTGGAGTGGGTCTCA |
| | nucleic acid | GGTGTAAGTATTAGGGGCATTGCCACATACTACGCAGACTCCGTGAAGGGCCGGTTCACCATCTCCA |
| | | GGGACGATTCCAGGAACACGTTGTATCTACAAATGAACAGCCTGAGAGCCGAGGACACGGCCCTTTA |
| | | TTACTGTGCGACCGGTGTTAGACTCCTTGGGCGTGAGTGGGGCCAGGGAACCCTGGTCACCGTCTCC |
| | | TCG |
| SEQ ID NO: 4 | Heavy chain | GFGLRWVFLVAILKGVQCEVQLLESGGGLVQPGGSLRLSCAASGFTFSNFGMTWVRQAPGKGLEW |
| | variable region | VSGVSIRGIATYYADSVKGRFTISRDDSRNTLYLQMNSLRAEDTALYYCATGVRLLGREWGQGTLV |
| | 2B9 protein | TVSS |
| SEQ ID NO: 5 | CDR1 Heavy chain- | GFTFSNFG |
| | protein 2B9 | |
| SEQ ID NO: 6 | CDR2 Heavy chain- | VSIRGIAT |
| | protein 2B9 | |
| SEQ ID NO: 7 | CDR3 Heavy chain- | CATGVRLLGREW |
| | protein 2B9 | |
| SEQ ID NO: 8 | Light chain 2B9 | ATGAGGCTCCCTGCTCAGCTCCTGGGGCTGCTAATGCTCTGGGTCCCAGGTTCCAGTGGGGATGT |
| | nucleic acid | TGTGATGACTCAGTCTCCACTCTCCCTGCCCGTCCCCCTTGGACAGCCGGCCTCCATCTCCTGCA |
| | | GGTCTAGTCAAAGCCTCGTCGACAATGATGGAAACACGTACTTGAATTGGTTTCAGCAGAGGCCA |
| | | GGCCAATCTCCAAGGCGCCTAATTTATAAGGTTTCTATGCGGGACTCTGGGGTCCCAGACAGATT |
| | | CACCGGCAGTGGGTCAGGCACTGATTTCACACTGAGGATTAGCAGGGTGGAGGCTGACGATGTTG |
| | | GAGTTTATTACTGCATGCAAGGTACACACTGGCCTCTCACTTTCGGCCCTGGGACCAAAGTGGAG |
| | | ATCAAACGAACTGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAAGTACAGTGGA |
| | | AGGTGGATAACGCCCTCCAATCGGGTAACTCCCAGGAGAGTGTCACAGAGCAGGACAGCAAGGA |
| | | CAGCACCTACAGCCTCAGCAGCACCCTGACGCTGAGCAAAGCAGACTACGAGAAACACAAAGTC |
| | | TACGCCTGCGAAGTCACCCATCAGGGCCTGAGCTCGCCCGTCACAAAGAGCTTCAACAGGGG |
| | | AGAGTGTTAG |
| SEQ ID NO: 9 | Light chain 2B9 | DVVMTQSPLSLPVPLGQPASISCRSSQSLVDNDGNTYLNWFQQRP |
| | Protein (kappa | GQSPRRLIYKVSMRDSGVPDRFTGSGSGTDFTLRISRVEADDVGVYYCMQGTHWPLTFGPGTKVE |
| | chain) | IKRTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC |
| | | [Constant region is italicized] |
| SEQ ID NO: 10 | Light chain | ATGAGGCTCCCTGCTCAGCTCCTGGGGCTGCTAATGCTCTGGGTCCCAGGTTCCAGTGGGGATGTTG |
| | variable region | TGATGACTCAGTCTCCACTCTCCCTGCCCGTCCCCCTTGGACAGCCGGCCTCCATCTCCTGCAGGTC |
| | 2B9 nucleic acid | TAGTCAAAGCCTCGTCGACAATGATGGAAACACGTACTTGAATTGGTTTCAGCAGAGGCCAGGCCAA |
| | | TCTCCAAGGCGCCTAATTTATAAGGTTTCTATGCGGGACTCTGGGGTCCCAGACAGATTCACCGGCA |
| | | GTGGGTCAGGCACTGATTTCACACTGAGGATTAGCAGGGTGGAGGCTGACGATGTTGGAGTTTATTA |
| | | CTGCATGCAAGGTACACACTGGCCTCTCACTTTCGGCCCTGGGACCAAAGTGGAGATCAAACGA |
| SEQ ID NO: 11 | Light chain | DVVMTQSPLSLPVPLGQPASISCRSSQSLVDNDGNTYLNWFQQRP |
| | variable | GQSPRRLIYKVSMRDSGVPDRFTGSGSGTDFTLRISRVEADDVGVYYCMQGTHWPLTFGPGTKVE |
| | region protein | IKR |
| SEQ ID NO: 12 | CDR1 Light chain | QSLVDNDGNTY |
| | 2B9 protein | |
| SEQ ID NO: 13 | CDR2 Light chain | KVS |
| | 2B9 protein | |
| SEQ ID NO: 14 | CDR3 Light chain | CMQGTHWPLTF |
| | 2B9 protein | |
| SEQ ID NO: 15 | 2B9 scFv | FGLRWVFLVAILKGVQCEVQLLESGGGLVQPGGSLRLSCAASGFTFSNFGMTWVRQAPGKGLEWV |
| | | SGVSIRGIATYYADSVKGRFTISRDDSRNTLYLQMNSLRAEDTALYYCATGVRLLGREWGQGTLV |
| | | TVSSggggsggggsggggsDVVMTQSPLSLPVPLGQPASISCRSSQSLVDNDGNTYLNWFQQRPGQS |
| | | PRRLIYKVSMRDSGVPDRFTGSGSGTDFTLRISRVEADDVGVYYCMQGTHWPLTFGPGTKVEIKR |
| | | [Linker sequence is italicized] |
| SEQ ID NO: 16 | Human PDK1 | MARTTSQLYDAVPIQSSVVLCSCPSPSMVRTQTESSTPPGIPGGSRQGPAMDGTAAEPRP |
| | isoform 1 | GAGSLQHAQPPPQPRKKRPEDFKFGKILGEGSFSTVVLARELATSREYAIKILEKRHIIK |
| | protein | ENKVPYVTRERDVMSRLDHPFFVKLYFTFQDDEKLYFGLSYAKNGELLKYIRKIGSFDET |
| | | CTRFYTAEIVSALEYLHGKGIIHRDLKPENILLNEDMHIQITDFGTAKVLSPESKQARAN |
| | | SFVGTAQYVSPELLTEKSACKSSDLWALGCIIYQLVAGLPPFRAGNEYLIFQKIIKLEYD |
| | | FPEKFFPKARDLVEKLLVLDATKRLGCEEMEGYGPLKAHPFFESVTWENLHQQTPPKLTA |
| | | YLPAMSEDDEDCYGNYDNLLSQFGCMQVSSSSSSHSLSASDTGLPQRSGSNIEQYIHDLD |
| | | SNSFELDLQFSEDEKRLLLEKQAGGNPWHQFVENNLILKMGPVDKRKGLFARRRQLLLTE |
| | | GPHLYYVDPVNKVLKGEIPWSQELRPEAKNFKTFFVHTPNRTYYLMDPSGNAHKWCRKIQ |
| | | EVWRQRYQSHPDAAVQ |
| SEQ ID NO: 17 | Human PDK1 | MDGTAAEPRPGAGSLQHAQPPPQPRKKRPEDFKFGKILGEGSFSTVVLARELATSREYAI |
| | isoform 2 protein | KILEKRHIIKENKVPYVTRERDVMSRLDHPFFVKLYFTFQDDEKLYFGLSYAKNGELLKY |
| | | IRKIGSFDETCTRFYTAEIVSALEYLHGKGIIHRDLKPENILLNEDMHIQITDFGTAKVL |
| | | SPESKQARANSFVGTAQYVSPELLTEKSACKSSDLWALGCIIYQLVAGLPPFRAGNEYLI |
| | | FQKIIKLEYDFPEKFFPKARDLVEKLLVLDATKRLGCEEMEGYGPLKAHPFFESVTWENL |
| | | HQQTPPKLTAYLPAMSEDDEDCYGNYDNLLSQFGCMQVSSSSSSHSLSASDTGLPQRSGS |
| | | NIEQYIHDLDSNSFELDLQFSEDEKRLLLEKQAGGNPWHQFVENNLILKMGPVDKRKGLF |
| | | ARRRQLLLTEGPHLYYVDPVNKVLKGEIPWSQELRPEAKNFKTFFVHTPNRTYYLMDPSG |
| | | NAHKWCRKIQEVWRQRYQSHPDAAVQ |
| SEQ ID NO: 18 | Human PDK1 | MARTTSQLYDAVPIQSSVVLCSCPSPSMVRTQTESSTPPGIPGGSRQGPAMDGTAAEPRP |
| | isoform 3 protein | GAGSLQHAQPPPQPRKKRPEDFKFGKILGEGSFSTVVLARELATSREYAIKILEKRHIIK |
| | | ENKVPYVTRERDVMSRLDHPFFVKLYFTFQDDEKLYFGLSYAKNGELLKYIRKIGSFDET |
| | | CTRFYTAEIVSALEYLHGKGIIHRDLKPENILLNEDMHIQITDFGTAKVLSPESKQADLW |
| | | ALGCIIYQLVAGLPPFRAGNEYLIFQKIIKLEYDFPEKFFPKARDLVEKLLVLDATKRLG |
| | | CEEMEGYGPLKAHPFFESVTWENLHQQTPPKLTAYLPAMSEDDEDCYGNYDNLLSQFGCM |
| | | QVSSSSSSHSLSASDTGLPQRSGSNIEQYIHDLDSNSFELDLQFSEDEKRLLLEKQAGGN |
| | | PWHQFVENNLILKMGPVDKRKGLFARRRQLLLTEGPHLYYVDPVNKVLKGEIPWSQELRP |
| | | EAKNFKTFFVHTPNRTYYLMDPSGNAHKWCRKIQEVWRQRYQSHPDAAVQ |
| SEQ ID NO: 19 | Human PDK1 | MARTTSQLYDAVPIQSSVVLCSCPSPSMVRTQTESSTPPGIPGGSRQGPAMDGTAAEPRP |
| | isoform 4 protein | GAGSLQHAQPPPQPRKKRPEDFKFGKILGEGSFSTVVLARELATSREYATRANSFVGTAQ |
| | | YVSPELLTEKSACKSSDLWALGCIIYQLVAGLPPFRAGNEYLIFQKIIKLEYDFPEKFFP |
| | | KARDLVEKLLVLDATKRLGCEEMEGYGPLKAHPFFESVTWENLHQQTPPKLTAYLPAMSE |
| | | DDEDCYGNYDNLLSQFGCMQVSSSSSSHSLSASDTGLPQRSGSNIEQYIHDLDSNSFELD |
| | | LQFSEDEKRLLLEKQAGGNPWHQFVENNLILKMGPVDKRKGLFARRRQLLLTEGPHLYYV |
| | | DPVNKVLKGEIPWSQELRPEAKNFKTFFVHTPNRTYYLMDPSGNAHKWCRKIQEVWRQRY |
| | | QSHPDAAVQ |
| SEQ ID NO: 20 | Human PDK1 | MARTTSQLYDAVPIQSSVVLCSCPSPSMVRTQTESSTPPGIPGGSRQGPAMDGTAAEPRP |
| | isoform 5 protein | GAGSLQHAQPPPQPRKKRPEDFKFGKILGEGSFSTVVLARELATSREYAIKILEKRHIIK |
| | | ENKVPYVTRERDVMSRLDHPFFVKLYFTFQDDEKLYFGLSYAKNGELLKYIRKIGSFDET |
| | | CTRFYTAEIVSALEYLHGKGIIHRDLKPENILLNEDMHIQITDFGTAKVLSPESKQARAN |
| | | SFVGTAQYVSPELLTEKSACKSSDLWALGCIIYQLVAGLPPFRAGNEYLIFQKIIKLEYD |
| | | FPEKFFPKARDLVEKLLVLDATKRLGCEEMEGYGPLKAHPFFESVTWENLHQQTPPKLTA |
| | | YLPAMSEDDEDCYGNYDNLLSQFGCMQVSSSSSSHSLSASDTGLPQRSGSNIEQYIHDLD |
| | | SNSFELDLQFSEDEKRLLLEKQAGGNPCLTGRII |
| SEQ ID NO: 21 | Linker sequence | GGGGSGGGGSGGGGS |

1. A binding molecule which binds to extracellular 3-phosphoinositide-dependent protein kinase-1 (PDK1).

2. The binding molecule of claim 1, which binds to a cell expressing extracellular 3-phosphoinositide-dependent protein kinase-1 (PDK1) and does not bind to a cell which does not express extracellular PDK1.

3. The binding molecule of claim 1, which is an antigen binding protein.

4. The binding molecule of claim 1, which is an antibody, or antigen binding fragment thereof.

5. The binding molecule of claim 4, wherein the antibody, or antigen binding fragment thereof, comprises a heavy chain variable region comprising a CDR3 having the amino acid sequence of SEQ ID NO: 7 and a light chain variable region comprising a CDR3 having the amino acid sequence of SEQ ID NO: 14.

6. The binding molecule of claim 5, wherein the antibody, or antigen binding fragment thereof, comprises a heavy chain variable region comprising a CDR2 having the amino acid sequence of SEQ ID NO: 6 and a light chain variable region comprising a CDR2 having the amino acid sequence of SEQ ID NO: 13.

7. The binding molecule of claim 6, wherein the antibody, or antigen binding fragment thereof, comprises a heavy chain variable region comprising a CDR1 having the amino acid sequence of SEQ ID NO: 5 and a light chain variable region comprising a CDR1 having the amino acid sequence of SEQ ID NO: 12.

8. The binding molecule of claim 4, wherein the antibody, or antigen binding fragment thereof, comprises a heavy chain variable region comprising the amino acid sequence of SEQ ID NO: 4.

9. The binding molecule of claim 4, wherein the antibody, or antigen binding fragment thereof, comprises a light chain variable region comprising the amino acid sequence of SEQ ID NO: 11.

10. The binding molecule of claim 9, wherein the antibody, or antigen binding fragment thereof, comprises a heavy chain variable region comprising the amino acid sequence set forth in SEQ ID NO: 4.

11. A pharmaceutical composition comprising the binding molecule of claim 1, and a pharmaceutically acceptable carrier.

12. A pharmaceutical composition comprising the binding molecule of claim 10, and a pharmaceutically acceptable carrier.

13. A method of treating a cancer in a subject in need thereof comprising administering to the subject the binding molecule of claim 1, thereby treating the cancer in the subject.

14. A method of treating a cancer in a subject in need thereof comprising:
(a) obtaining a sample from a subject having cancer;
(b) testing the sample for expression of extracellular 3-phosphoinositide-dependent protein kinase-1(PDK1); and
(c) administering to the subject a therapeutically effective amount of a PDK1 inhibitor.

15. The method of claim 13, wherein the cancer is metastatic or non-resectable.

16. The method of claim 13, wherein the cancer is selected from the group consisting of breast cancer, lung cancer, prostate cancer, acute myeloid leukemia (AML), cervical cancer and squamous cell carcinoma.

17. The method of claim 13, further comprising administering to the subject at least one additional therapeutic agent.

18. A method for determining malignancy of a tumor from a subject comprising determining the presence of extracellular 3-phosphoinositide-dependent protein kinase-1 (PDK1) in a sample from a tumor from the subject by contacting the sample with the binding molecule of claim 1, wherein the presence of extracellular PDK1 as detected by the binding molecule in the sample indicates that the tumor is malignant.

19. A method of identifying a subject having cancer which is responsive to the binding molecule of claim 1, said method comprising determining the presence of extracellular 3-phosphoinositide-dependent protein kinase-1 (PDK1) in a sample from the subject, wherein the presence of extracellular PDK1 in the sample indicates that cancer will be responsive to treatment with the antigen binding protein.

20. A method of detecting extracellular 3-phosphoinositide-dependent protein kinase-1 (PDK1) in a sample, comprising contacting the sample with the binding molecule of claim 1.

21. A method for reducing cell migration and invasion, the method comprising the step of contacting a cell expressing extracellular PDK1 with the binding molecule of claim 1, such that cell migration and invasion is reduced.

22. A method for reducing PDK1 activity, the method comprising the step of contacting a cell expressing extracellular PDK1 with the a binding molecule of claim 1, such that PDK activity is reduced.

23. The method of claim 21, wherein the method is in vivo.

24. A nucleic acid encoding a chimeric antigen receptor (CAR), wherein the CAR comprises an extracellular binding domain capable of binding to extracellular PDK1, a transmembrane domain, and an intracellular signaling domain.

25. The nucleic acid of claim 24, wherein the extracellular binding domain is an anti-PDK1 antibody, or an antigen binding fragment thereof.

26. The nucleic acid of claim 25, wherein the antibody, or antigen binding fragment thereof, comprises a heavy chain variable region comprising a CDR1 having an amino acid sequence of SEQ ID NO: 5, a CDR2 having an amino acid sequence of SEQ ID NO: 6, and CDR3 having an amino acid sequence of SEQ ID NO: 7, and comprises a light chain variable region comprising a CDR1 having an amino acid sequence of SEQ ID NO: 12, a CDR2 having an amino acid sequences of SEQ ID NO: 13, and CDR3 having an amino acid sequence of SEQ ID NO: 14.

27. The nucleic acid of claim 25, wherein the antibody, or antigen binding fragment thereof, comprises a heavy chain variable region comprising an amino acid sequence of SEQ ID NO: 4, and a light chain variable region comprising an amino acid sequence of SEQ ID NO: 11.

28. The nucleic acid of claim 27, wherein the antigen binding fragment is an scFv.

29. A vector comprising the nucleic acid sequence of claim 24.

30. An immune effector cell comprising the vector of claim 29.

31. A method of treating cancer in a subject in need thereof, comprising administering to the subject the immune effector cell of claim 30.

32. An isolated anti-PDK1 human antibody, or antigen binding portion thereof, produced by hybridoma cell line 2B9 (ATCC Accession No. ______).

33. The hybridoma cell line 2B9 deposited under ATCC Accession No. ______.


SEQUENCE LISTING (21 sequences)

SEQ ID NO: 1 - DNA, 759 bp, Artificial Sequence
  Description of Artificial Sequence Synthetic polynucleotide
ggatttgggctgcgctgggtttttcttgtggctattttaaaaggtgtccagtgtgaggtgcagctgttggagtcagggggaggcttggtacagccgggggggtccctgagactctcctgtgcagcctctgggttcacctttagtaactttggcatgacctgggtccgccaggctccggggaaggggctggagtgggtctcaggtgtaagtattaggggcattgccacatactacgcagactccgtgaagggccggttcaccatctccagggacgattccaggaacacgttgtatctacaaatgaacagcctgagagccgaggacacggccctttattactgtgcgaccggtgttagactccttgggcgtgagtggggccagggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctg

SEQ ID NO: 2 - protein, 253 aa, Artificial Sequence
  Description of Artificial Sequence Synthetic polypeptide
GFGLRWVFLVAILKGVQCEVQLLESGGGLVQPGGSLRLSCAASGFTFSNFGMTWVRQAPGKGLEWVSGVSIRGIATYYADSVKGRFTISRDDSRNTLYLQMNSLRAEDTALYYCATGVRLLGREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELL

SEQ ID NO: 3 - DNA, 405 bp, Artificial Sequence
  Description of Artificial Sequence Synthetic polynucleotide
ggatttgggctgcgctgggtttttcttgtggctattttaaaaggtgtccagtgtgaggtgcagctgttggagtcagggggaggcttggtacagccgggggggtccctgagactctcctgtgcagcctctgggttcacctttagtaactttggcatgacctgggtccgccaggctccggggaaggggctggagtgggtctcaggtgtaagtattaggggcattgccacatactacgcagactccgtgaagggccggttcaccatctccagggacgattccaggaacacgttgtatctacaaatgaacagcctgagagccgaggacacggccctttattactgtgcgaccggtgttagactccttgggcgtgagtggggccagggaaccctggtcaccgtctcctcg

SEQ ID NO: 4 - protein, 135 aa, Artificial Sequence
  Description of Artificial Sequence Synthetic polypeptide
GFGLRWVFLVAILKGVQCEVQLLESGGGLVQPGGSLRLSCAASGFTFSNFGMTWVRQAPGKGLEWVSGVSIRGIATYYADSVKGRFTISRDDSRNTLYLQMNSLRAEDTALYYCATGVRLLGREWGQGTLVTVSS

SEQ ID NO: 5 - protein, 8 aa, Artificial Sequence
  Description of Artificial Sequence Synthetic peptide
GFTFSNFG

SEQ ID NO: 6 - protein, 8 aa, Artificial Sequence
  Description of Artificial Sequence Synthetic peptide
VSIRGIAT

SEQ ID NO: 7 - protein, 12 aa, Artificial Sequence
  Description of Artificial Sequence Synthetic peptide
CATGVRLLGREW

SEQ ID NO: 8 - DNA, 720 bp, Artificial Sequence
  Description of Artificial Sequence Synthetic polynucleotide
atgaggctccctgctcagctcctggggctgctaatgctctgggtcccaggttccagtggggatgttgtgatgactcagtctccactctccctgcccgtcccccttggacagccggcctccatctcctgcaggtctagtcaaagcctcgtcgacaatgatggaaacacgtacttgaattggtttcagcagaggccaggccaatctccaaggcgcctaatttataaggtttctatgcgggactctggggtcccagacagattcaccggcagtgggtcaggcactgatttcacactgaggattagcagggtggaggctgacgatgttggagtttattactgcatgcaaggtacacactggcctctcactttcggccctgggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 9 - protein, 219 aa, Artificial Sequence
  Description of Artificial Sequence Synthetic polypeptide
DVVMTQSPLSLPVPLGQPASISCRSSQSLVDNDGNTYLNWFQQRPGQSPRRLIYKVSMRDSGVPDRFTGSGSGTDFTLRISRVEADDVGVYYCMQGTHWPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 399 bp, Artificial Sequence
  Description of Artificial Sequence Synthetic polynucleotide
atgaggctccctgctcagctcctggggctgctaatgctctgggtcccaggttccagtggggatgttgtgatgactcagtctccactctccctgcccgtcccccttggacagccggcctccatctcctgcaggtctagtcaaagcctcgtcgacaatgatggaaacacgtacttgaattggtttcagcagaggccaggccaatctccaaggcgcctaatttataaggtttctatgcgggactctggggtcccagacagattcaccggcagtgggtcaggcactgatttcacactgaggattagcagggtggaggctgacgatgttggagtttattactgcatgcaaggtacacactggcctctcactttcggccctgggaccaaagtggagatcaaacga

SEQ ID NO: 11 - protein, 113 aa, Artificial Sequence
  Description of Artificial Sequence Synthetic polypeptide
DVVMTQSPLSLPVPLGQPASISCRSSQSLVDNDGNTYLNWFQQRPGQSPRRLIYKVSMRDSGVPDRFTGSGSGTDFTLRISRVEADDVGVYYCMQGTHWPLTFGPGTKVEIKR

SEQ ID NO: 12 - protein, 11 aa, Artificial Sequence
  Description of Artificial Sequence Synthetic peptide
QSLVDNDGNTY

SEQ ID NO: 13 - protein, 3 aa, Artificial Sequence
  Description of Artificial Sequence Synthetic peptide
KVS

SEQ ID NO: 14 - protein, 11 aa, Artificial Sequence [sequence omitted]

SEQ ID NO: 15 - protein, 262 aa, Artificial Sequence
  Description of Artificial Sequence Synthetic polypeptide
FGLRWVFLVAILKGVQCEVQLLESGGGLVQPGGSLRLSCAASGFTFSNFGMTWVRQAPGKGLEWVSGVSIRGIATYYADSVKGRFTISRDDSRNTLYLQMNSLRAEDTALYYCATGVRLLGREWGQGTLVTVSSGGGGSGGGGSGGGGSDVVMTQSPLSLPVPLGQPASISCRSSQSLVDNDGNTYLNWFQQRPGQSPRRLIYKVSMRDSGVPDRFTGSGSGTDFTLRISRVEADDVGVYYCMQGTHWPLTFGPGTKVEIKR

SEQ ID NO: 16 - protein, 556 aa, Homo sapiens
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 17 - protein, 506 aa, Homo sapiens
MDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 18 - protein, 530 aa, Homo sapiens
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQADLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 19 - protein, 429 aa, Homo sapiens
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYATRANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 20 - protein, 454 aa, Homo sapiens
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPCLTGRII

SEQ ID NO: 21 - protein, 15 aa, Artificial Sequence
  Description of Artificial Sequence Synthetic peptide
GGGGSGGGGSGGGGS